(19) United States
(12) Patent Application Publication
(10) Pub. No.: US 2023/0158104 A1
(43) Pub. Date: May 25, 2023

(54) COMPOUNDS FOR USE IN AUTOIMMUNE CONDITIONS

(71) Applicant: PHARMA MAR, S.A., Madrid (ES)

(72) Inventors: Pablo AVILÉS MARÍN, Madrid (ES); Alejandro LOSADA GONZÁLEZ, Madrid (ES); José María FERNÁNDEZ SOUSA-FARO, Madrid (ES)

(21) Appl. No.: 17/908,532

(22) PCT Filed: Mar. 2, 2020
(86) PCT No.: PCT/EP2021/055142

§ 371 Date: Aug. 31, 2022

(30) Foreign Application Priority Data
Mar. 2, 2020 (EP) 20382152.5
Mar. 13, 2020 (EP) 20382192.1
Apr. 2, 2020 (EP) 20382266.3
Apr. 27, 2020 (EP) 20382339.8
Sep. 16, 2020 (EP) 20382814.0
Sep. 16, 2020 (EP) 20382815.7
Jan. 25, 2021 (EP) 21382059.0

(51) Int. Cl. A61K 38/15; A61P 31/16

(52) U.S. Cl. CPC A61K 38/15; A61P 31/16

(57) ABSTRACT

The present invention relates to the use of compounds in the treatment of autoimmune conditions, and in particular, for the treatment of rheumatoid arthritis.

### FIELD OF THE INVENTION

The present invention relates to the treatment of autoimmune conditions.

### BACKGROUND TO THE INVENTION

Autoimmune conditions are characterised by chronic inflammation, implicated in which is the activation of the Toll-like receptor (TLR) pathway. In this pathway, harmful stimuli triggered by injury, infection, stress, hypoxia or cell death all ignite tissue damage and lead to the release of endogenous TLR ligands or “auto-antigens”. There are multiple hypotheses as to how such endogenous TLR ligands are generated during autoimmunity to result in inflammation, and it is possible that many or all of these processes are occurring during autoimmune disease.

One hypothesis is that antigens that are usually intracellular and therefore not visible to the immune system may accumulate on the membranes of cells due to high levels of apoptosis, becoming visible to the immune system and acting as a TLR ligand. Another hypothesis is that neo-epitopes capable of inducing an immune response including acting as TLR ligands may be generated. Neo-epitopes may be generated through the modification of existing molecules by enzymatic cleavage, post-translational modifications or other structural modifications. These changes may be triggered by an environmental factor or by an in vivo change e.g. dysregulation of enzyme activity, such as increased granzyme B activity due to apoptosis.

These ligands, also known as DAMPs (damage-associated molecular patterns) bind to a Toll-like receptor (of which there are 10 sub-types in humans, named TLR1-10) leading to activation of signalling cascades that culminate in an inflammatory response. Endogenous TLR ligands have been identified for at least TLRs 2, 3, 4, 5, 7, 8, 9 and 11 and are linked to a number of autoimmune diseases. In addition, microbial products, which are known TLR ligands have also been found in patients suffering from autoimmune disease and, in addition to endogenous TLR ligands, can drive TLR signalling cascades.

One such signalling cascade results in the activation of the canonical NF-κB pathway, which is the pivotal regulator of inflammation and the central mediator of pro-inflammatory gene induction, and therefore a key driver of autoimmune pathology.

In the NF-κB pathway, binding of endogenous ligands to TLRs triggers receptor dimerization. Downstream, TLRs are capable of interacting with a series of adaptor proteins that mediate different signaling pathways. Myeloid differentiation primary response protein 88 (MyD88) is the most widely utilised TLR adaptor protein and mediates signaling through all TLRs. MyD88 interacts with the threonine-serine kinase interleukin (IL)-1 receptor-associated kinase 4 (IRAK4), which upon activation phosphorylates IRAK1. Subsequently, the IRAKs recruit the ubiquitin ligase tumor necrosis factor receptor-associated factor 6 (TRAF-6), which polyubiquitinates and activates TAK1 kinase. TAK1 kinase activates the IKK complex that triggers the proteolytic degradation of inhibitor κB (I-κB), the inhibitor of nuclear factor κB (NF-κB), which unmasks the nuclear localization signal of NF-κB allowing translocation of this transcription complex from the cytoplasm to the nucleus and activation of a wide variety of NF-κB responsive genes, including genes encoding proinflammatory cytokines and co-stimulatory molecules required for activation of the adaptive immune response.

As such, activation of NF-κB transduction is responsible for the transcriptional induction of pro-inflammatory cytokines, chemokines and additional inflammatory mediators in different types of immune cells. These inflammatory mediators can both directly engage in the induction of inflammation and act indirectly through promoting the differentiation of inflammatory T cells. In this way, activation or dysregulation of TLR signalling leads to chronic inflammation, which is central to the pathogenesis of autoimmune conditions.

There therefore exists a need to develop new therapies for the treatment of autoimmune conditions, which at present is untreatable in the majority of patients affected. In particular, there is a need to develop a therapy that can halt TLR activation or prevent pathology, such as autoimmune conditions, arising from TLR activation. The present invention addresses these needs.

### SUMMARY OF THE INVENTION

In one aspect, the present invention is directed to a compound of general formula I, or a pharmaceutically acceptable salt or stereoisomer thereof,

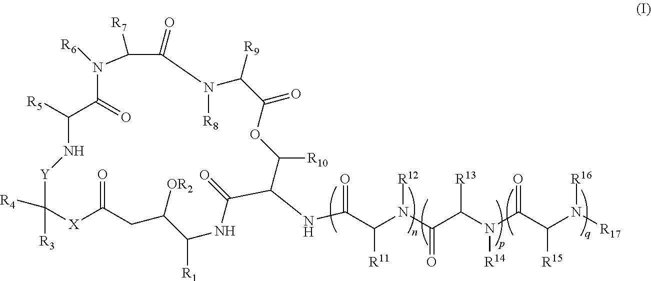

wherein X is selected from O and NH;

Y is selected from CO and —COCH(CH₃)CO—;

each n and p is independently selected from 0 and 1, and q is selected from 0, 1 and 2;

each R1, R3, R5, R9, R11, and R15 is independently selected from hydrogen, substituted or unsubstituted C1-C6 alkyl, substituted or unsubstituted C2-C6 alkenyl, and substituted or unsubstituted C2-C6 alkynyl;

R2 is selected from hydrogen, CORa, COORa, substituted or unsubstituted C1-C6 alkyl, substituted or unsubstituted C2-C6 alkenyl, and substituted or unsubstituted C2-C6 alkynyl;

each R4, R8, R10, R12, and R16 is independently selected from hydrogen and substituted or unsubstituted C1-C6 alkyl;

each R7 and R13 is independently selected from hydrogen, substituted or unsubstituted C1-C6 alkyl, substituted or unsubstituted C2-C6 alkenyl, and substituted or unsubstituted C2-C6 alkynyl; each R6 and R14 is independently selected from hydrogen and substituted or unsubstituted C1-C6 alkyl; or R6 and R7 and/or R13 and R14 together with the corresponding N atom and C atom to which they are attached may form a substituted or unsubstituted heterocyclic group;

R17 is selected from hydrogen, CORa, COORa, CONHRb, COSRc, (C═NRb)ORa, (C═NRb)NHRb, (C═NRb)SRc, (C═S)ORa, (C═S)NHRb, (C═S)SRc, SO2Rc, SO3Rc, substituted or unsubstituted C1-C12 alkyl, substituted or unsubstituted C2-C12 alkenyl, substituted or unsubstituted C2-C12 alkynyl, substituted or unsubstituted aryl, and substituted or unsubstituted heterocyclic group, with the proviso that when n, p, and q are 0 then R17 is not hydrogen; and

each Ra, Rb, and Rc is independently selected from hydrogen, substituted or unsubstituted C1-C12 alkyl, substituted or unsubstituted C2-C12 alkenyl, substituted or unsubstituted C2-C12 alkynyl, substituted or unsubstituted aryl, and substituted or unsubstituted heterocyclic group;

for use in the treatment of an autoimmune condition.

In a particular aspect, the compound of general formula I is PLD, or a pharmaceutically acceptable salt or stereoisomer thereof.

In another aspect, the present invention is directed to DidemninB, or a pharmaceutically acceptable salt or stereoisomer thereof.

In another aspect, the present invention is also directed to a pharmaceutical composition comprising a compound as defined herein, and a pharmaceutically acceptable carrier, for use according to the present invention.

In another aspect, the present invention is directed to the use of a compound as defined herein, in the manufacture of a medicament for the treatment of an autoimmune condition.

In another aspect, the present invention is directed to a method for treating any mammal, preferably a human, for an autoimmune condition, wherein the method comprises administering to an individual in need thereof a therapeutically effective amount of a compound as defined herein.

In embodiments, the autoimmune condition is selected from systemic lupus erythematosus (SLE), rheumatoid arthritis (RA), multiple sclerosis (MS), scleroderma, Sjögren's syndrome, autoimmune myocarditis, type 1 diabetes, and atherosclerosis. In a preferred embodiment, the autoimmune condition is RA.

In a further aspect of the invention, there is provided a kit comprising the compound as defined herein, or a pharmaceutically acceptable salt or stereoisomer thereof, together with instructions for treating an autoimmune condition.

The following embodiments apply to all aspects of the present invention.

The autoimmune condition may be caused by the activation of one or more Toll-like receptor (TLR).

The autoimmune condition may be characterised by increased signalling through at least one or more Toll-like receptor (TLR).

The autoimmune condition may be characterised by increased levels of at least one pro-inflammatory cytokines.

The autoimmune condition may be selected from systemic lupus erythematosus (SLE), rheumatoid arthritis (RA), multiple sclerosis (MS), scleroderma, Sjögren's syndrome, autoimmune myocarditis, type 1 diabetes, and atherosclerosis. In a preferred embodiment, the autoimmune condition is RA.

R₃ and R₄ may be independently selected from hydrogen and substituted or unsubstituted C₁-C₆ alkyl. R₃ may be isopropyl and R₄ may be hydrogen. R₃ and R₄ may be methyl (this compound is also designated a compound of general formula II).

R₁₁ may be selected from hydrogen and substituted or unsubstituted C₁-C₆ alkyl. R₁₁ may be methyl or isobutyl. R₁₁ may be methyl and n=1 (this compound is also designated a compound of general formula III).

R₁, R₅, R₉, and R₁₅ may be independently selected from hydrogen and substituted or unsubstituted C₁-C₆ alkyl. R₁ may be selected from sec-butyl and isopropyl, R₅ may be isobutyl, R₉ may be p-methoxybenzyl, and R₁₅ may be selected from methyl and benzyl.

R₈, R₁₀, R₁₂, and R₁₆ may be independently selected from hydrogen and substituted or unsubstituted C₁-C₆ alkyl. R₈, R₁₀ and R₁₂ may be methyl, and R₁₆ may be hydrogen.

R₆ and R₁₄ may be independently selected from hydrogen and substituted or unsubstituted C₁-C₆ alkyl. R₆ may be selected from hydrogen and methyl, and R₁₄ may be hydrogen.

R₇ and R₁₃ may be independently selected from hydrogen and substituted or unsubstituted C₁-C₆ alkyl. R₇ may be methyl and R₁₃ may be selected from hydrogen, methyl, isopropyl, isobutyl, and 3-amino-3-oxopropyl.

R₆ and R₇ and/or R₁₃ and R₁₄ together with the corresponding N atom and C atom to which they are attached may form a substituted or unsubstituted pyrrolidine group.

R₂ may be selected from hydrogen, substituted or unsubstituted C₁-C₆ alkyl, and CORₐ, and wherein Rₐ may be a substituted or unsubstituted C₁-C₆ alkyl. R₂ may be hydrogen.

R₁₇ may be selected from hydrogen, CORₐ, COORₐ, CONHR_{b}, (C═S)NHR_{b}, and SO₂R_{c}, and wherein each Rₐ, R_{b}, and R_{c} may be independently selected from substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group. R₁₇ may be selected from hydrogen, COObenzyl, CObenzo[b]thiophen-2-yl, SO₂(p-methylphenyl), COCOCH₃ and COOC(CH₃)₃.

X may be NH. X may be O. Y may be CO. Y may be —COCH(CH₃)CO—.

The compound may be PLD, or pharmaceutically acceptable salts or stereoisomers thereof. The compound may be PLD.

The compound may be didemninB, or pharmaceutically acceptable salts or stereoisomers thereof.

The compound may be didemninB.

### DESCRIPTION OF THE FIGURES

The invention is further described in the following non-limiting figures:

FIG. 1 shows that NF-κB transactivation in response to the activation of Toll-like receptors is inhibited by PLD. Human monocytic cells (THP-1) were stably transfected with a NF-kB-Luc plasmid and (A) levels of NF-kB transactivation measured in the presence and absence of PLD. (B) Compound-induced cytotoxicity was tested by the MTT cell proliferation assay. Cultures were exposed to PLD at 100 nM for 6 hours. RQ—Resiquimod at 10 μg/mL. LPS-B5-Lipopolysaccharide from Escherichia coli 055:B5 (LPS-B5) at 10 μg/mL. Poly-C—Polyinosinic-polycytidylic at 500 μg/mL. TNF-α was used as a positive control. ***p<0.001; **p<0.01

FIG. 2 shows that NF-κB transactivation in response to the activation of Toll-like receptors leads to increased secretion of the pro-inflammatory cytokines: IL-1, IL-6, IL-8 and TNF-α. Cultures were exposed to PLD at 100 nM or DMSO for 6 hours. At 6 hours post-treatment secreted cytokines were analysed by ELISA. TNF-α was used as a positive control. ***p<0.001; **p<0.01

FIG. 3 shows the ex-vivo down-regulation of cytokines IL-6, IL-10 and TNF-α by PLD.

FIG. 4 shows a decrease in classically activated macrophages in LPS-challenged mice.

FIG. 5 shows x-rays showing the effects of PLD administration on a patient with bilateral pneumonia.

FIG. 6 shows x-rays showing the effects of PLD administration on a patient with unilateral pneumonia.

FIG. 7 shows C-reactive protein tests for patients treated with PLD.

FIG. 8 shows the inflammatory profile in the BALF of mice infected with influenza virus with (PR8) or without (PC) treatment with PLD.

FIG. 9 shows the effect of plitidepsin (APL) pre-treatment at 1 nM, 10 nM and 50 nM on secretion of the pro-inflammatory cytokines IL6 (a), IL8 (b), IL1β (c) and TNF-α (d). (e) shows the effect of 1 nM, 10 nM and 50 nM PLD treatment on cell viability (as a percent of control). At 0 time THP-1 cells were treated with 1 nM, 10 nM or 50 nM APL or DMSO (0.2%) followed by stimulus with Resiquimod at 2.5 or 5 μg/mL at 8 hours. At 24 hours cytokines or cell viability was measured.

FIG. 10 shows the effect of plitidepsin treatment on the production of the pro-inflammatory cytokines, IL-6 (c), IL-10 (d) and TNF-α (e) mediated by LPS-B5 in CD45⁺ cells isolated from bronco-alveolar lavages (BALF). (a) shows the percent of CD45⁺ live cells in control, LPS-B5 and LPS-B5 and PLD treated cells. (b) shows cell survival as a percent of control in LPS-B5 and LPS-B5 and PLD treated cells.

FIG. 11 shows the effect of plitidepsin treatment on the production of the pro-inflammatory cytokines TNF-α mediated by Resiquimod.

FIG. 12 shows the effects of plitidepsin on alveolar macrophage recruitment in LPS treated mice. Concentration-time curves (mean±SD) of plitidepsin in plasma and lung of mice (a), rats (b) and hamsters (c) after a single intravenous dose at 1.0, 0.2 and 0.2 mg/kg respectively.

### DETAILED DESCRIPTION OF THE INVENTION

The following embodiments apply to all aspects of the present invention.

The present invention will now be further described. In the following passages, different aspects of the invention are defined in more detail. Each aspect so defined may be combined with any other aspect or aspects or embodiment or embodiments unless clearly indicated to the contrary.

In particular, any feature indicated as being preferred or advantageous may be combined with any other feature or features indicated as being preferred or advantageous.

In the present application, a number of general terms and phrases are used, which should be interpreted as follows.

The term “treating”, as used herein, unless otherwise indicated, means reversing, attenuating, alleviating or inhibiting the progress of the disease or condition to which such term applies, or one or more symptoms of such disorder or condition. The term treating as used herein may also include prophylactic treatment, that is treatment designed to prevent the autoimmune condition from occurring or minimize the likelihood of an autoimmune condition occurring.

“Patient” includes humans, non-human mammals (e.g., dogs, cats, rabbits, cattle, horses, sheep, goats, swine, deer, and the like) and non-mammals (e.g., birds, and the like).

Plitidepsin (PLD) is a cyclic depsipeptide originally isolated from the marine tunicate Aplidium albicans. PLD is also known as Aplidin. PLD analogues are those analogues as defined herein.

In a preferred embodiment, the present invention relates to the use of PLD.

We have found that PLD
(i) inhibits transactivation of NF-κB induced by activation of Toll-like receptors;
(ii) inhibits secretion of pro-inflammatory cytokines, such as IL-1, IL-6, IL-8 and TNF-α both in vivo and ex vivo; and
(iii) inhibits activation of macrophages.

These properties means that PLD has particular efficacy in the treatment of autoimmune conditions. Reference to PLD herein can be considered applicable to the compounds of the invention (other PLD analogues). As shown in the Examples, we have found that PLD can inhibit the secretion of pro-inflammatory cytokines, thereby reducing levels of inflammation, which is the main contributor to the pathogenesis of autoimmune conditions. In particular, we have found that PLD can inhibit the transactivation of NF-kB through the Toll-like receptors (TLR) and subsequent secretion of pro-inflammatory cytokines. For example, we have shown that PLD can inhibit the transactivation of NF-kB through the activation of TLR3, TLR4, TL7 and TLR8, all of which have been shown to be activated by endogenous ligands.

As explained herein, in autoimmune conditions, the Toll-like receptors are activated in response to a number of endogenous ligands that are released from damaged tissues. Binding of TLR ligands (i.e. stimuli) to a Toll-like receptor TLR triggers a downstream signalling cascade that ultimately leads to the activation of the transcription factor nuclear factor-kappa B (NF-kB), which controls induction of pro-inflammatory cytokines and chemokines. We have found that PLD significantly blocks this cascade, consequently leading to a reduction in the release of pro-inflammatory cytokines. As a result, in one example, PLD can be used to prevent an autoimmune condition following activation of the Toll-like receptors.

We have also found that PLD significantly reduces levels of macrophage activation and/or macrophage recruitment. Activated macrophages are a key mediator of inflammation and inhibition of macrophage activation is central to treating inflammation and thus the pathology of an autoimmune condition.

Accordingly, compounds as defined herein (including PLD and DidemninB), particularly PLD, can be used in the treatment autoimmune condition following activation of the Toll-like receptors.

The present invention may be useful in relation to the following autoimmune conditions:

Rheumatoid Arthritis (RA)

Rheumatoid arthritis (RA) is a chronic inflammatory autoimmune condition characterised by the progressive and irreversible destruction of joints. RA is the most common autoimmune condition, affecting around 1% of the population. At present, there is no cure, and up to 40% of the population does not respond to existing therapies. RA is characterised by persistent inflammation driven by the proliferating synovial tissue fibroblasts, as well as T and B cells, neutrophils and monocytes trafficking into the joints. A variety of endogenous TLR ligands, including fibrinogen, HSP60, 70, EDA fibronectin, HMGB1, hyaluronate and HSP22, have been demonstrated to be present within the inflamed joints of patients with RA, and have been shown to lead to activation of the TLRs: TLR2, TLR4, TLR5 and TLR7. Activation of these TLRs have all been implicated to be responsible for the persistent expression of pro-inflammatory cytokines and activated macrophages observed in RA joints, with the different TLR family members being implicated at different stages of the disease. Consistent with the activation of TLR in the pathogenesis of RA, activated NF-kB has also been detected in human synovial tissue at both early and late stages of the disease, and is believed to be responsible for both the initiation and the perpetuation of chronic inflammation seen in RA. In particular, many of these ligands are thought to be induced by cellular injury, extracellular matrix degradation and activated macrophage activity, which are all hallmark features of RA. Therefore, the RA microenvironment may facilitate sustained and worsening disease by further release of these ligands. Interestingly, fragments of double stranded viral RNA released by necrotic cells is an effective TLR ligand and has been found in the synovial fluid of RA patients, supporting the hypothesis that microbial infection may trigger or sustain TLR responses in RA, causing disease to form and flare.

Systemic Lupus Erythematosus (SLE)

Systemic lupus erythematosus (SLE) or lupus is a severe, relapsing, remitting autoimmune condition that causes a number of symptoms in affected patients, including joint pain, skin rashes and tiredness. In some cases the disease also affects the kidneys as well as other organs. Patient sera has been found to contain ligands for the TLRs, and in particular, TLR7, TLR8 and TLR9. Peripheral dendritic cells are recognised as key drivers of RA pathology, and these cells express both TLR7 and TLR9 meaning they can be activated by such ligands to cause disease relevant signalling. In particular, in patients with SLE, autoreactive cells produce large quantities of autoantibodies against self-nuclear antigens, making immune complexes with self-nucleic acids in the serum. These complexes act as TLR ligands, particularly for TLR7 and TLR9, activating the TLR pathway and causing chronic inflammation.

Similarly to RA, single stranded viral RNA has also been detected in lupus patients, as well as those suffering from other autoimmune diseases such as scleroderma and Sjögren's syndrome, and are effective ligands for TLRs 7 and 8. Bacterial or HSV DNA has also been found in lupus patients and is an effective ligand of TLR9. Numerous experimental systems have now demonstrated that microbial TLR ligands are able to cause disease in experimental models of arthritis, multiple sclerosis, experimental allergic encephalomyelitis (EAE), autoimmune myocarditis, type 1 diabetes and atherosclerosis. Once again, this widely supports a microbial involvement in TLR activation driving autoimmune disease.

Multiple Sclerosis (MS)

Multiple Sclerosis (MS) is an autoimmune condition in which CNS lesions result from perivascular immune cell infiltration associated with damage to myelin, oligodendrocytes and neurons. Clinically, symptoms include numbness, weakness, loss of muscle coordination, and problems with vision, speech, and bladder control. MS pathology comprises two main phases, firstly an initial immune activation where an autoimmune response is triggered, and secondly, recruitment of immune cells into the CNS where tissue destruction and demyelination occurs. Studies indicate that TLRs play a significant role in modulating MS, as well as experimental autoimmune encephalomyelitis (EAE), an animal model of MS. Interestingly, as well as recruited immune cells, it has been found that resident microglia in the CNS also express a range of TLRs and that expression of these TLRs is increased in response to inflammatory mediators. These cells have been shown to be vital to establishing and worsening inflammatory plaques in the CNS during MS, and it is likely that they are propagating disease through progressive activation of these receptors.

Scleroderma

Scleroderma or systemic sclerosis, is a chronic connective tissue disease generally classified as an autoimmune rheumatic diseases. Scleroderma is caused by the immune system attacking the connective tissue under the skin and around internal organs and blood vessels. This causes scarring and thickening of the tissue in these areas. Some types of scleroderma are relatively mild and may eventually improve on their own, while others can lead to severe and life-threatening problems for which there is no cure. TLRs have been identified as critical in the pathogenesis of scleroderma where products from damaged cells, i.e. endogenous TLR ligands, trigger TLR signalling which drives inflammatory and fibrotic activity. In particular, it is thought that TLR signalling can drive the release of TIMPs to cause fibrosis in scleroderma.

Sjögren's Syndrome

Sjögren's syndrome is an autoimmune disorder that often co-exists with RA and/or lupus, and primarily affects the salivary and lacrimal glands. These glands help the body create moisture in the eyes and mouth, in the form of saliva and tears. Thus, in a person with Sjögren's syndrome, the body fails to produce enough moisture. It is thought that TLRs may underlie this disorder, with a number of putative endogenous TLR ligands found in patients with Sjögren's syndrome or mice models of the disease, including as bigylcan, decorin, versican and fibronectin. It has also been found that TLR expression is upregulated and is hyper-responsive to ligation on peripheral blood cells from patients suffering from Sjögren's syndrome.

Autoimmune Myocarditis

Autoimmune myocarditis is an autoimmune disease that affects the heart. The condition is characterized by inflammation of the heart muscle, and does not affect any other organ. Again, it appears that TLR signalling is an important underlying mechanism to myocarditis pathology. For example, knockout mice for MyD88, which is a canonical adapter molecule that facilitates downstream TLR signalling, are protected from disease in an induced model of myocarditis. It has been postulated that human cardiac myosin may act as an endogenous TLR ligand in order to trigger downstream pro-inflammatory responses through TLR2 and TLR8.

Type 1 Diabetes

Type 1 diabetes, or insulin-dependent diabetes, is an autoimmune disease that causes the insulin producing beta cells in the pancreas to be destroyed. This results in the patient only being able to produce very small amounts of insulin, or not being able to produce any insulin at all, which is a hormone that is required for the effective control of blood sugar. It has been shown that viral infection may cause this cellular destruction through TLR9 induced immune activation, and that upregulation of TLRs can increase disease penetrance. This demonstrates an important role for TLR signalling in the pathogenesis of type 1 diabetes.

Atherosclerosis

Atherosclerosis is condition where the build-up of fats, cholesterol and other substances in and on the artery walls (plaque), can restrict blood flow. These plaques can burst, triggering a blood clot and causing related conditions such as stroke or myocardial infarction, and in particular driving cardiovascular disease (CVD). Atherosclerosis is now considered to be an inflammatory autoimmune condition, and in particular it is thought that TLRs are key orchestrators of the disease process. There is a plethora of evidence that supports this from disease models, including knockouts of MyD88, TLR2 and TLR4 all being able to reduce or prevent atherosclerosis in mouse models. It is thought that TLR2 and TLR4 are active during disease and can be activated by a range of lipopeptides (Falck-Hansen et al, 2013).

In view of the above, it is clear that TLR signalling is an underlying driver of autoimmunity, whether this be in response to endogenous TLR ligands, microbial TLR ligands or a combination of both, and that in many cases, the disease microenvironment can facilitate a positive feedback loop to sustain TLR signalling.

Accordingly, compounds of the present invention (including PLD) can be used in the treatment of autoimmune conditions.

In these compounds the groups can be selected in accordance with the following guidance:

Alkyl groups may be branched or unbranched, and preferably have from 1 to about 12 carbon atoms. One more preferred class of alkyl groups has from 1 to about 6 carbon atoms. Even more preferred are alkyl groups having 1, 2, 3 or 4 carbon atoms. Methyl, ethyl, n-propyl, isopropyl and butyl, including n-butyl, tert-butyl, sec-butyl and isobutyl are particularly preferred alkyl groups in the compounds of the present invention. As used herein, the term alkyl, unless otherwise stated, refers to both cyclic and noncyclic groups, although cyclic groups will comprise at least three carbon ring members.

Preferred alkenyl and alkynyl groups in the compounds of the present invention may be branched or unbranched, have one or more unsaturated linkages and from 2 to about 12 carbon atoms. One more preferred class of alkenyl and alkynyl groups has from 2 to about 6 carbon atoms. Even more preferred are alkenyl and alkynyl groups having 2, 3 or 4 carbon atoms. The terms alkenyl and alkynyl as used herein, unless otherwise stated, refer to both cyclic and noncyclic groups, although cyclic groups will comprise at least three carbon ring members.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Preferably aryl groups contain from 6 to about 10 carbon ring atoms. Specially preferred aryl groups include substituted or unsubstituted phenyl, substituted or unsubstituted naphthyl, substituted or unsubstituted biphenyl, substituted or unsubstituted phenanthryl, and substituted or unsubstituted anthryl.

Suitable heterocyclic groups include heteroaromatic and heteroalicyclic groups containing from 1 to 3 separated or fused rings and from 5 to about 18 ring atoms. Preferably heteroaromatic and heteroalicyclic groups contain from 5 to about 10 ring atoms, most preferably 5, 6 or 7 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl including pyrazol-3-yl, pyrazol-4-yl and pyrazol-5-yl, pyrimidinyl, furanyl including furan-2-yl, furan-3-yl, furan-4-yl and furan-5-yl, pyrrolyl, thienyl, thiazolyl including thiazol-2-yl, thiazol-4-yl and thiazol-5-yl, isothiazolyl, thiadiazolyl including thiadiazol-4-yl and thiadiazol-5-yl, triazolyl, tetrazolyl, isoxazolyl including isoxazol-3-yl, isoxazol-4-yl and isoxazol-5-yl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl including benzo[b]thiophen-2-yl and benzo[b]thiophen-3-yl, benzothiazolyl, benzoxazolyl, imidazo[1,2-a]pyridinyl including imidazo[1,2-a]pyridine-2-yl and imidazo[1,2-a]pyridine-3-yl, quinazolinyl, quinoxalinyl, naphthyridinyl and furopyridyl. Suitable heteroalicyclic groups in the compounds of the present invention contain one, two or three heteroatoms selected from N, O or S atoms and include, e.g., pyrrolidinyl, tetrahydrofuranyl, dihydrofuranyl, tetrahydrothienyl, tetrahydrothiopyranyl, piperidinyl including piperidin-3-yl, piperidin-4-yl and piperidin-5-yl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridyl, 2-pyrrolinyl, 3-pyrrolinyl, dihydropyrrolyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

In the above mentioned groups one or more hydrogen atoms may be substituted by one or more suitable groups such as OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. When a substituent group terminates with a double bound (such as ═O and ═N—R′) it replaces 2 hydrogen atoms in the same carbon atom.

Suitable halogen substituents in the compounds of the present invention include F, Cl, Br and I.

The term “pharmaceutically acceptable salts” refers to any salt which, upon administration to the patient is capable of providing (directly or indirectly) a compound as described herein. It will be appreciated that non-pharmaceutically acceptable salts also fall within the scope of the invention since those may be useful in the preparation of pharmaceutically acceptable salts. The preparation of salts can be carried out by methods known in the art. For instance, pharmaceutically acceptable salts of compounds provided herein are synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts.

The compounds of the invention may be in crystalline form either as free compounds or as solvates (e.g. hydrates, alcoholates, particularly methanolates) and it is intended that both forms are within the scope of the present invention. Methods of solvation are generally known within the art. The compounds of the invention may present different polymorphic forms, and it is intended that the invention encompasses all such forms

Any compound referred to herein is intended to represent such specific compound as well as certain variations or forms. In particular, compounds referred to herein may have asymmetric centres and therefore exist in different enantiomeric or diastereomeric forms. Thus any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same or different than the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

In compounds of general formula I and II, particularly preferred R₁, R₅, R₉, R₁₁, and R₁₅ are independently selected from hydrogen and substituted or unsubstituted C₁-C₆ alkyl. More preferred R₁, R₅, R₉, R₁₁, and R₁₅ are independently selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, methyl, n-propyl, isopropyl, isobutyl, sec-butyl, 4-aminobutyl, 3-amino-3-oxopropyl, benzyl, p-methoxybenzyl, p-hydroxybenzyl, and cyclohexylmethyl are the most preferred R₁, R₅, R₉, R₁₁, and R₁₅ groups. Specifically, particularly preferred R₁ is selected from sec-butyl and isopropyl, being sec-butyl the most preferred. Particularly preferred R₅ is selected from isobutyl and 4-aminobutyl, being isobutyl the most preferred. Particularly preferred R₁₁ is methyl and isobutyl. Particularly preferred R₉ is selected from p-methoxybenzyl, p-hydroxybenzyl, and cyclohexylmethyl, being p-methoxybenzyl the most preferred. Particularly preferred R₁₅ is selected from methyl, n-propyl, and benzyl, being methyl and benzyl the most preferred.

In compounds of general formula III, particularly preferred R₁, R₅, R₉, and R₁₅ are independently selected from hydrogen and substituted or unsubstituted C₁-C₆ alkyl. More preferred R₁, R₅, R₉, and R₁₅ are independently selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, methyl, n-propyl, isopropyl, isobutyl, sec-butyl, 4-aminobutyl, 3-amino-3-oxopropyl, benzyl, p-methoxybenzyl, p-hydroxybenzyl, and cyclohexylmethyl are the most preferred R₁, R₅, R₉, and R₁₅ groups. Specifically, particularly preferred R₁ is selected from sec-butyl and isopropyl, being sec-butyl the most preferred. Particularly preferred R₅ is selected from isobutyl and 4-aminobutyl, being isobutyl the most preferred. Particularly preferred R₉ is selected from p-methoxybenzyl, p-hydroxybenzyl, and cyclohexylmethyl, being p-methoxybenzyl the most preferred. Particularly preferred R₁₅ is selected from methyl, n-propyl, and benzyl, being methyl and benzyl the most preferred.

In compounds of general formula I, II and III, particularly preferred R₈, R₁₀, R₁₂, and R₁₆ are independently selected from hydrogen and substituted or unsubstituted C₁-C₆ alkyl. More preferred R₈, R₁₀, R₁₂, and R₁₆ are independently selected from hydrogen, methyl, ethyl, n-propyl, isopropyl and butyl, including n-butyl, tert-butyl, isobutyl and sec-butyl, and even more preferred they are independently selected from hydrogen and methyl. Specifically, particularly preferred R₈, R₁₀ and R₁₂ are methyl, and particularly preferred R₁₆ is hydrogen.

In compounds of general formula I and III, particularly preferred R₃ and R₄ are independently selected from hydrogen and substituted or unsubstituted C₁-C₆ alkyl. More preferred R₃ and R₄ are independently selected from hydrogen, substituted or unsubstituted methyl, substituted or unsubstituted ethyl, substituted or unsubstituted n-propyl, substituted or unsubstituted isopropyl, and substituted or unsubstituted butyl, including substituted or unsubstituted n-butyl, substituted or unsubstituted tert-butyl, substituted or unsubstituted isobutyl and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, methyl, isopropyl, and sec-butyl are the most preferred R₃ and R₄ groups. Specifically, particularly preferred R₃ is selected from methyl and isopropyl and particularly preferred R₄ is methyl or hydrogen.

In one embodiment of compounds of general formula I, II and III, particularly preferred R₆ and R₇ are independently selected from hydrogen and substituted or unsubstituted C₁-C₆ alkyl. More preferred R₇ is selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. More preferred R₆ is selected from hydrogen, methyl, ethyl, n-propyl, isopropyl and butyl, including n-butyl, tert-butyl, isobutyl and sec-butyl. Most preferred R₆ is selected from hydrogen and methyl and most preferred R₇ is methyl.

In another embodiment of compounds of general formula I, II and III, it is particularly preferred that R₆ and R₇ together with the corresponding N atom and C atom to which they are attached form a substituted or unsubstituted heterocyclic group. In this regard, preferred heterocyclic group is a heteroalicyclic group containing one, two or three heteroatoms selected from N, O or S atoms, most preferably one N atom, and having from 5 to about 10 ring atoms, most preferably 5, 6 or 7 ring atoms. A pyrrolidine group is the most preferred.

In one embodiment of compounds of general formula I, II and III, particularly preferred R₁₃ and R₁₄ are independently selected from hydrogen and substituted or unsubstituted C₁-C₆ alkyl. More preferred R₁₃ is selected from hydrogen, substituted or unsubstituted methyl, substituted or unsubstituted ethyl, substituted or unsubstituted n-propyl, substituted or unsubstituted isopropyl and substituted or unsubstituted butyl, including substituted or unsubstituted n-butyl, substituted or unsubstituted tert-butyl, substituted or unsubstituted isobutyl, and substituted or unsubstituted sec-butyl. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. More preferred R₁₄ is selected from hydrogen, methyl, ethyl, n-propyl, isopropyl and butyl, including n-butyl, tert-butyl, isobutyl and sec-butyl. Most preferred R₁₃ is selected from hydrogen, methyl, isopropyl, isobutyl, and 3-amino-3-oxopropyl and most preferred R₁₄ is hydrogen.

In another embodiment of compounds of general formula I, II and III, it is particularly preferred that R₁₃ and R₁₄ together with the corresponding N atom and C atom to which they are attached form a substituted or unsubstituted heterocyclic group. In this regard, preferred heterocyclic group is a heteroalicyclic group containing one, two or three heteroatoms selected from N, O or S atoms, most preferably one N atom, and having from 5 to about 10 ring atoms, most preferably 5, 6 or 7 ring atoms. A pyrrolidine group is the most preferred.

In compounds of general formula I, II and III, particularly preferred R₂ is selected from hydrogen, substituted or unsubstituted C₁-C₆ alkyl, and CORₐ, wherein Rₐ is a substituted or unsubstituted C₁-C₆ alkyl, and even more preferred Rₐ is methyl, ethyl, n-propyl, isopropyl and butyl, including n-butyl, tert-butyl, sec-butyl and isobutyl. More preferably R₂ is hydrogen.

In compounds of general formula I, II and III, particularly preferred R₁₇ is selected from hydrogen, CORₐ, COORₐ, CONHR_{b}, (C═S)NHR_{b}, and SO₂R_{c}, wherein each Rₐ, R_{b}, and R_{c} is preferably and independently selected from substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group. Preferred substituents of said groups are OR′, ═O, SR′, SOR′, SO₂R′, NO₂, NHR′, NR′R′, ═N—R′, NHCOR′, N(COR′)₂, NHSO₂R′, NR′C(═NR′)NR′R′, CN, halogen, COR′, COOR′, OCOR′, OCONHR′, OCONR′R′, CONHR′, CONR′R′, substituted or unsubstituted C₁-C₁₂ alkyl, substituted or unsubstituted C₂-C₁₂alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, CO₂H, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chosen from the foregoing list. Hydrogen, CORₐ, COORₐ, and SO₂R_{c} are the most preferred R₁₇ groups, and hydrogen, COObenzyl, CObenzo[b]thiophen-2-yl, SO₂(p-methylphenyl), COCOCH₃ and COOC(CH₃)₃ are even most preferred.

In another embodiment of compounds of general formula I, II and III, it is particularly preferred that Y is CO. In another embodiment, it is particularly preferred that Y is —COCH(CH₃)CO—.

In another embodiment of compounds of general formula I, II and III, it is particularly preferred that X is O. In another embodiment, it is particularly preferred that X is NH.

In another embodiment of compounds of general formula I and II, it is particularly preferred that n, p and q are 0. In another embodiment, it is particularly preferred that n is 1 and p and q are 0.

In another embodiment, it is particularly preferred that n and p are 1 and q is 0. In another embodiment, it is particularly preferred that n, p, and q are 1. In another embodiment, it is particularly preferred that n and p are 1 and q is 2.

In another embodiment of compounds of general formula III, it is particularly preferred that p and q are 0. In another embodiment, it is particularly preferred that p is 1 and q is 0. In another embodiment, it is particularly preferred that p and q are 1. In another embodiment, it is particularly preferred that p is 1 and q is 2.

In additional preferred embodiments, the preferences described above for the different substituents are combined. The present invention is also directed to such combinations of preferred substitutions of formula I, II and III above.

In the present description and definitions, when there are several groups Rₐ, R_{b}, and R_{c} present in the compounds of the invention, and unless it is stated explicitly so, it should be understood that they can be each independently different within the given definition, i.e. Rₐ does not represent necessarily the same group simultaneously in a given compound of the invention.

In compounds of general formula I, II and III when q takes a value of 2 there are two groups R₁₅ and two groups R₁₆ in the compound. It is hereby clarified that each R₁₅ and each R₁₆ group in a given compound may be independently selected among the different possibilities described above for such groups.

A particularly preferred stereochemistry for compounds of general formula I is

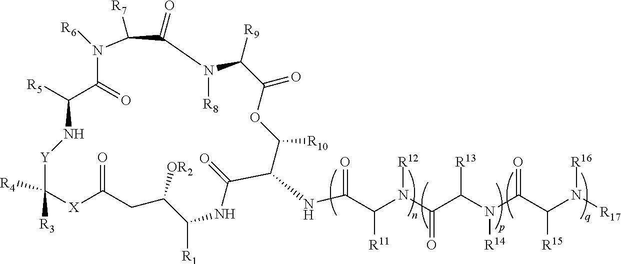

wherein X, Y, n, p, q, and R₁-R₁₇ are as defined above, and when Y is —COCH(CH₃)CO— it has the following stereochemistry:

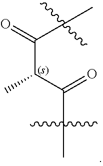

A particularly preferred stereochemistry for compounds of general formula II is

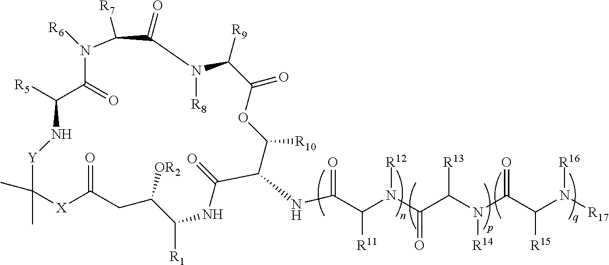

wherein X, Y, n, p, q, R₁, R₂, and R₅-R₁₇ are as defined above, and when Y is —COCH(CH₃)CO— it has the following stereochemistry:

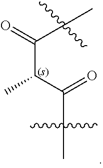

A particularly preferred stereochemistry for compounds of general formula III is

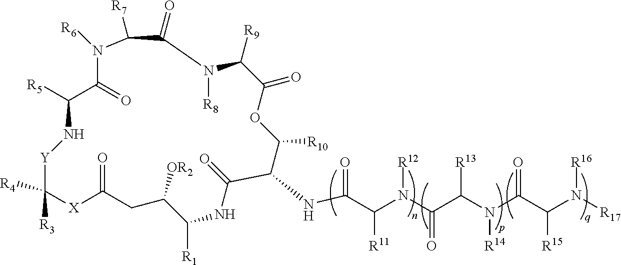

wherein X, Y, p, q, R₁-R₁₀, and R₁₂-R₁₇ are as defined above, and when Y is —COCH(CH₃)CO— it has the following stereochemistry:

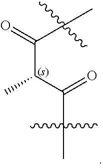

Particularly preferred compounds of the invention are the following:

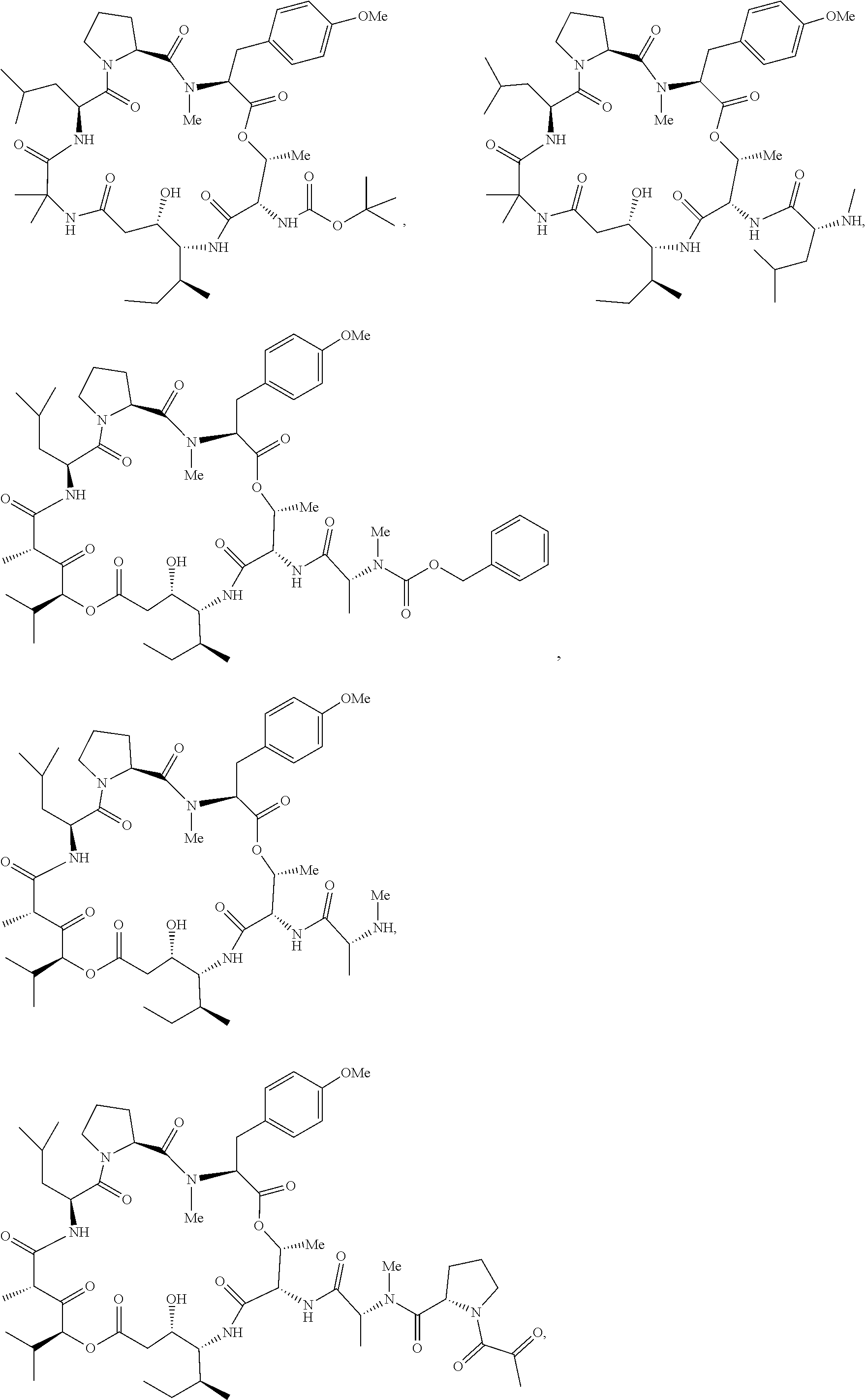

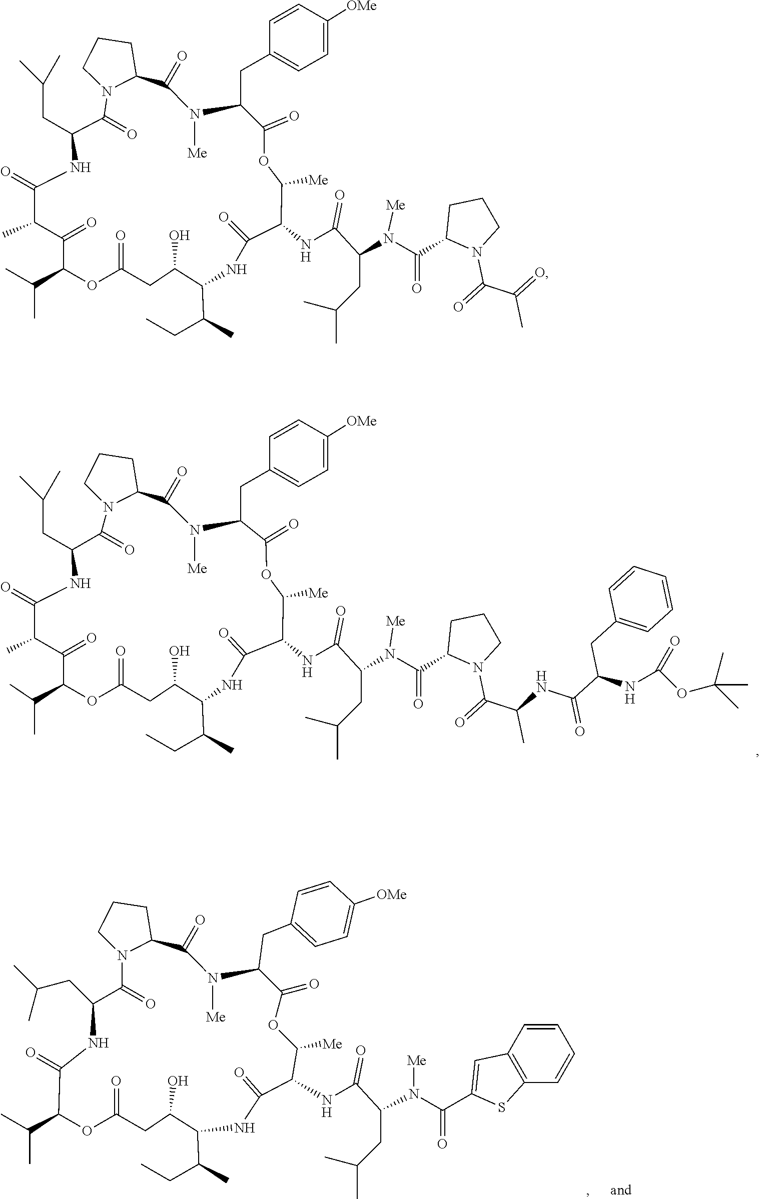

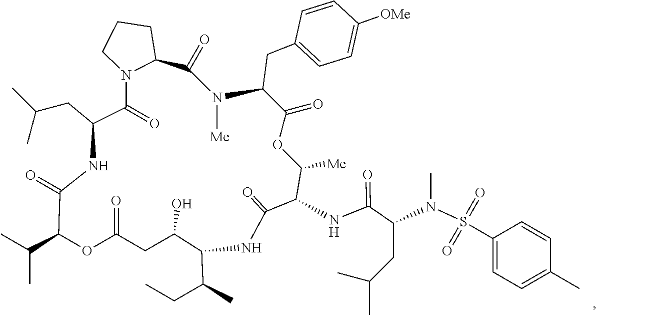 [SMILES: CC[C@H](C)[C@H]1NC(=O)[C@@H](NC(=O)[C@@H](CC(C)C)N(C)S(=O)(=O)c2ccc(C)cc2)[C@@H](C)OC(=O)[C@H](Cc2ccc(OC)cc2)N(C)C(=O)[C@@H]2CCCN2C(=O)[C@H](CC(C)C)NC(=O)[C@H](C(C)C)OC(=O)C[C@@H]1O]

or pharmaceutically acceptable salts or stereoisomers thereof.

The compounds of general formula I, II and III may be prepared following any of the synthetic processes disclosed in Vera et al. Med. Res. Rev. 2002, 22(2), 102-145, WO 2011/020913 (see in particular Examples 1-5), WO 02/02596, WO 01/76616, and WO 2004/084812, which are incorporated herein by reference.

The preferred compound is PLD or pharmaceutically acceptable salts or stereoisomers thereof. Most preferred is PLD.

The chemical name of plitidepsin is (−)-(3S,6R,7S,10R,11S,15S,17S,20S,25aS)-11-hydroxy-3-(4-methoxybenzyl)-2,6,17-trimethyl-15-(1-methylethyl)-7-[[(2R)-4-methyl-2-[methyl[[(2S)-1-(2-oxopropanoyl)pyrrolidin-2-yl]carbonyl]amino]pentanoyl]amino]-10-[(1S)-1-methylpropyl]-20-(2-methylpropyl)tetradecahydro-15H-pyrrolo[2,1-f]-[1,15,4,7,10,20]dioxatetrazacyclotricosine-1,4,8,13,16,18,21(17H)-heptone corresponding to the molecular formula C₅₇H₈₇N₇O₁₅. It has a relative molecular mass of 1110.34 g/mol and the following structure:

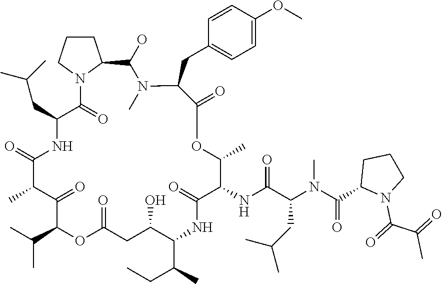 [SMILES: CC[C@H](C)[C@H]1NC(=O)[C@@H](NC(=O)[C@@H](CC(C)C)N(C)C(=O)[C@@H]2CCCN2C(=O)C(C)=O)[C@@H](C)OC(=O)[C@H](Cc2ccc(OC)cc2)N(C)C(O)[C@@H]2CCCN2C(=O)[C@H](CC(C)C)NC(=O)[C@@H](C)C(=O)[C@H](C(C)C)OC(=O)C[C@@H]1O]

Reference to general formula I, II and III includes reference to PLD and DidemninB. In preferred embodiments, the compound is PLD or Didemnin B. Most preferred is PLD.

The present invention provides the use of a compound as defined herein and pharmaceutically acceptable salts or stereoisomers thereof in the treatment of an autoimmune condition.

In one aspect of the invention, there is provided a compound of the present invention, for use in the treatment an autoimmune condition. In another aspect of the invention, there is provided the use of a compound of the present invention, in the manufacture of a medicament for the treatment of an autoimmune condition. In another aspect of the invention, there is provided a method for the treatment of an autoimmune condition, the method comprising administering to an individual in need thereof a therapeutically effective amount of a compound of the present invention.

In one embodiment, the autoimmune condition is caused by the activation of one or more Toll-like receptor (TLR) and/or is characterised by increased signalling through at least one TLR. Increased signalling through TLRs may be caused by an increase in expression in at least one TLR. In a further embodiment, the autoimmune condition is caused or contributed to by TLR-induced cytokine expression. In one embodiment, the TLR is TLR-3, TLR4, TLR7 or TLR8. Methods for measuring activation of TLR signalling in response to a known or possible TLR agonist would be well-known to the skilled person, but in one example, levels of NF-κB transactivation may be used as an indicator of TLR activation. As described herein NF-κB transactivation may be measured using luciferase-tagged NF-κB transactivation as described in the Examples. In another example, TLR activation can be determined by measuring any one of IRAK1 (IL-receptor-associated kinase), IRAK4 phosphorylation and TAK1 activation (transforming growth factor-p-activated kinase-1). Other indicators of TLR activation would be known in the art (see for example, Kawai & Akira, 2007, which describes the TLR pathway).

In another embodiment, the autoimmune condition is characterised by increased levels of at least one pro-inflammatory cytokines, and preferably at least one of IL-1, IL-6, IL-8, IL-10, IL-12 and CCL-2, more preferably at least one of IL-1, IL-6 and IL-8.

In a further embodiment, the autoimmune condition is selected from rheumatoid arthritis (RA), multiple sclerosis (MS), systemic lupus erythematosus (SLE), scleroderma, sjögren's syndrome, autoimmune myocarditis, type 1 diabetes, or atherosclerosis. In a preferred embodiment, the autoimmune condition is RA.

Compounds of the invention may be used in pharmaceutical compositions having biological/pharmacological activity for the treatment of the above mentioned conditions. These pharmaceutical compositions comprise a compound of the invention together with a pharmaceutically acceptable carrier. The term “carrier” refers to a diluent, adjuvant, excipient or vehicle with which the active ingredient is administered. Suitable pharmaceutical carriers are described in “Remington's Pharmaceutical Sciences” by E. W. Martin, 1995. Examples of pharmaceutical compositions include any solid (tablets, pills, capsules, granules, etc.) or liquid (solutions, suspensions, emulsions, etc.) compositions for oral, topical or parenteral administration. Pharmaceutical compositions containing compounds of the invention may be delivered by liposome or nanosphere encapsulation, in sustained release formulations or by other standard delivery means.

An exemplary composition is in the form of powder for solution for infusion. For example, compositions as described in WO9942125. For example, a lyophilised preparation of a compound of the invention including water-soluble material and secondly a reconstitution solution of mixed solvents. A particular example is a lyophilised preparation of PLD and mannitol and a reconstitution solution of mixed solvents, for example PEG-35 castor oil, ethanol and water for injections. Each vial, for example may contain 2 mg of PLD. After reconstitution, each mL of reconstituted solution may contain: 0.5 mg of PLD, 158 mg of PEG-35 castor oil, and ethanol 0.15 mL/mL.

The specific dosage and treatment regimen for any particular patient may be varied and will depend upon a variety of factors, including the activity of the specific compound employed, the particular formulation being used, the mode of application, the age, body weight, general health, sex, diet, time of administration, rate of excretion, drug combination, reaction sensitivities, and the severity of the particular disease or condition being treated.

According to further embodiments, patients may be selected for treatment with compounds of the present invention based on clinical parameters and/or patient characteristics. Suitable parameters may be measurements disclosed in the present application.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof.

However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

### EXAMPLES

Compounds of the present invention can be obtained according to the processes set out in the literature, for example: Vera et al. Med. Res. Rev. 2002, 22(2), 102-145, WO 2011/020913 (see in particular Examples 1-5), WO 02/02596, WO 01/76616, and WO 2004/084812, the contents of which are incorporated herein by reference.

Particular compounds of the present invention are:

| Compound | Structure |
|---|---|
| PLD | 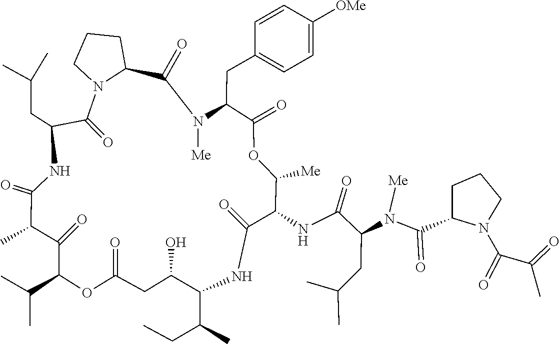 [SMILES: CC[C@H](C)[C@H]1NC(=O)[C@@H](NC(=O)[C@H](CC(C)C)N(C)C(=O)[C@@H]2CCCN2C(=O)C(C)=O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| DidemninB (compound 240) | 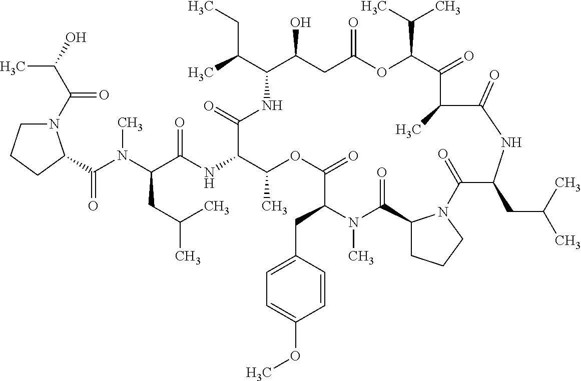 [SMILES: CC[C@H](C)[C@H]1NC(=O)[C@@H](NC(=O)[C@@H](CC(C)C)N(C)C(=O)[C@@H]2CCCN2C(=O)[C@H](C)O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| Compound 3 | 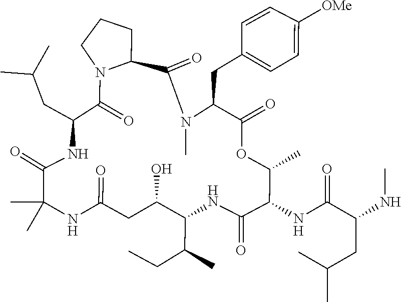 [SMILES: CC[C@H](C)[C@H]1NC(=O)[C@@H](NC(=O)[C@@H](CC(C)C)NC)[C@@H](C)OC(=O)[C@H](Cc2ccc(OC)cc2)N(C)C(=O)[C@@H]2CCCN2C(=O)[C@H](CC(C)C)NC(=O)C(C)(C)NC(=O)C[C@@H]1O] |
| Compound 8 | 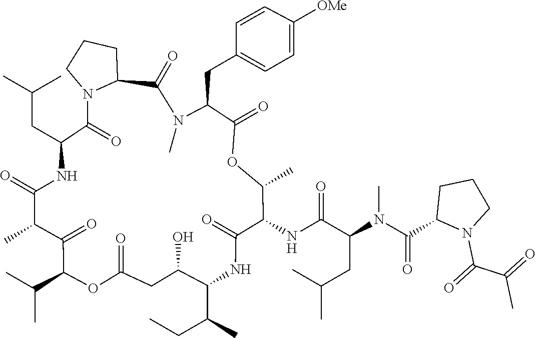 [SMILES: CC[C@H](C)[C@H]1NC(=O)[C@@H](NC(=O)[C@H](CC(C)C)N(C)C(=O)[C@@H]2CCCN2C(=O)C(C)=O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| Compound 9 | 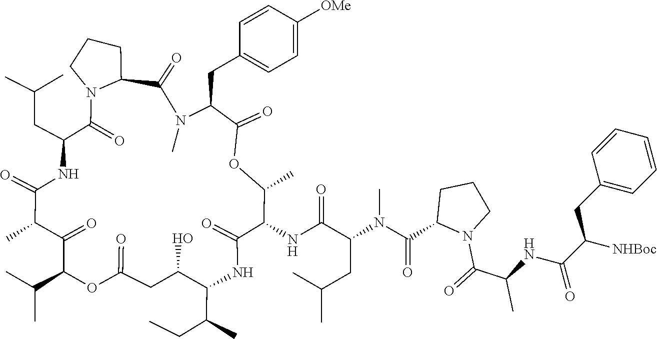 [SMILES: CC[C@H](C)[C@H]1NC(=O)[C@@H](NC(=O)[C@@H](CC(C)C)N(C)C(=O)[C@@H]2CCCN2C(=O)[C@H](C)NC(=O)[C@@H](Cc2ccccc2)NC(=O)OC(C)(C)C)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)OC(=O)C[C@@H]1O] |
| Compound 10 | 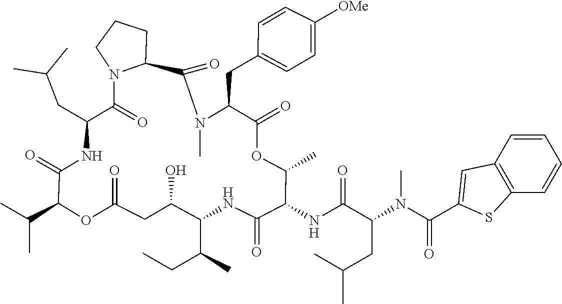 [SMILES: CC[C@H](C)[C@H]1NC(=O)[C@@H](NC(=O)[C@@H](CC(C)C)N(C)C(=O)c2cc3ccccc3s2)[C@@H](C)OC(=O)[C@H](Cc2ccc(OC)cc2)N(C)C(=O)[C@@H]2CCCN2C(=O)[C@H](CC(C)C)NC(=O)[C@H](C(C)C)OC(=O)C[C@@H]1O] |
| Compound 11 | 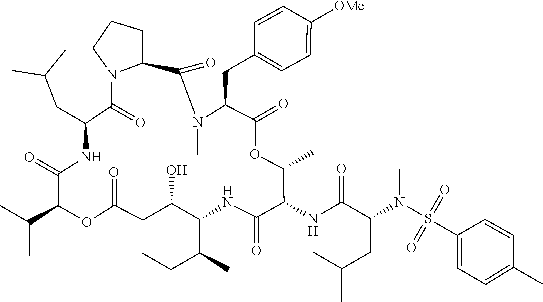 [SMILES: CC[C@H](C)[C@H]1NC(=O)[C@@H](NC(=O)[C@@H](CC(C)C)N(C)S(=O)(=O)c2ccc(C)cc2)[C@@H](C)OC(=O)[C@H](Cc2ccc(OC)cc2)N(C)C(=O)[C@@H]2CCCN2C(=O)[C@H](CC(C)C)NC(=O)[C@H](C(C)C)OC(=O)C[C@@H]1O] |

Following the procedures described in WO 02/02596 and in the specification, and further disclosed in the previous examples, the following compounds are obtainable:

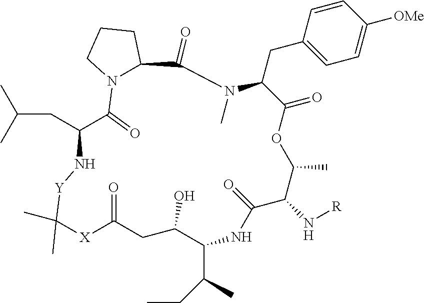

| Compound | X | Y | R |
|---|---|---|---|
| 12 13 14 15 | O NH O NH | CO CO —COCH(CH₃)CO— —COCH(CH₃)CO— | 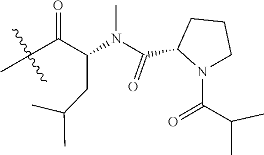 [SMILES: *C(=O)[C@@H](CC(C)C)N(C)C(=O)[C@@H]1CCCN1C(=O)C(C)C] |
| 16 17 18 19 | O NH O NH | CO CO —COCH(CH₃)CO— —COCH(CH₃)CO— | 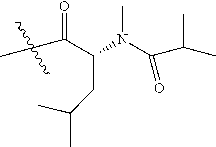 [SMILES: *C(=O)[C@@H](CC(C)C)N(C)C(=O)C(C)C] |
| 20 21 22 23 | O NH O NH | CO CO —COCH(CH₃)CO— —COCH(CH₃)CO— | 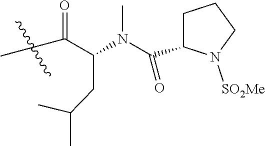 [SMILES: *C(=O)[C@@H](CC(C)C)N(C)C(=O)[C@@H]1CCCN1S(C)(=O)=O] |
| 24 25 26 27 | O NH O NH | CO CO —COCH(CH₃)CO— —COCH(CH₃)CO— | 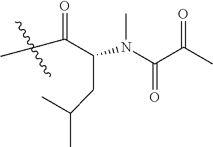 [SMILES: *C(=O)[C@@H](CC(C)C)N(C)C(=O)C(C)=O] |
| 28 29 30 31 | O NH O NH | CO CO —COCH(CH₃)CO— —COCH(CH₃)CO— | 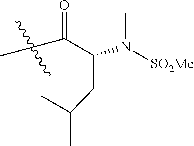 [SMILES: *C(=O)[C@@H](CC(C)C)N(C)S(C)(=O)=O] |
| 32 33 34 35 | O NH O NH | CO CO —COCH(CH₃)CO— —COCH(CH₃)CO— | 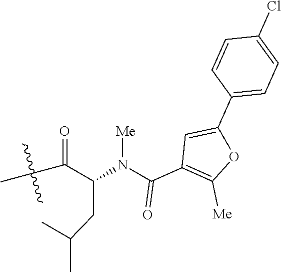 [SMILES: *C(=O)[C@@H](CC(C)C)N(C)C(=O)c1cc(-c2ccc(Cl)cc2)oc1C] |
| 36 37 38 39 | O NH O NH | CO CO —COCH(CH₃)CO— —COCH(CH₃)CO— | 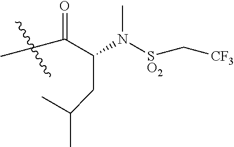 [SMILES: *C(=O)[C@@H](CC(C)C)N(C)S(=O)(=O)CC(F)(F)F] |
| 40 41 42 43 | O NH O NH | CO CO —COCH(CH₃)CO— —COCH(CH₃)CO— | 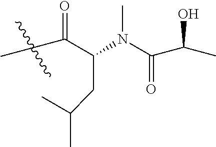 [SMILES: *C(=O)[C@@H](CC(C)C)N(C)C(=O)[C@H](C)O] |
| 44 45 46 47 | O NH O NH | CO CO —COCH(CH₃)CO— —COCH(CH₃)CO— | 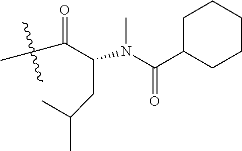 [SMILES: *C(=O)[C@@H](CC(C)C)N(C)C(=O)C1CCCCC1] |
| 48 49 50 51 | O NH O NH | CO CO —COCH(CH₃)CO— —COCH(CH₃)CO— | 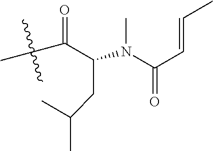 [SMILES: *C(=O)[C@@H](CC(C)C)N(C)C(=O)/C=C/C] |
| 52 53 54 55 | O NH O NH | CO CO —COCH(CH₃)CO— —COCH(CH₃)CO— | 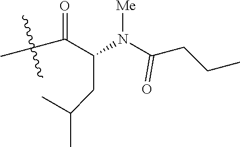 [SMILES: *C(=O)[C@@H](CC(C)C)N(C)C(=O)CCC] |
| 56 57 58 59 | O NH O NH | CO CO —COCH(CH₃)CO— —COCH(CH₃)CO— | 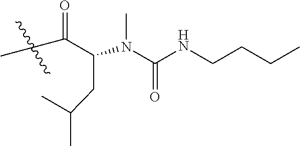 [SMILES: *C(=O)[C@@H](CC(C)C)N(C)C(=O)NCCCC] |
| 60 61 62 63 | O NH O NH | CO CO —COCH(CH₃)CO— —COCH(CH₃)CO— | 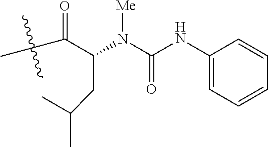 [SMILES: *C(=O)[C@@H](CC(C)C)N(C)C(=O)Nc1ccccc1] |
| 64 65 66 67 | O NH O NH | CO CO —COCH(CH₃)CO— —COCH(CH₃)CO— | 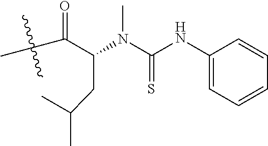 [SMILES: *C(=O)[C@@H](CC(C)C)N(C)C(=S)Nc1ccccc1] |
| 68 69 70 71 | O NH O NH | CO CO —COCH(CH₃)CO— —COCH(CH₃)CO— | 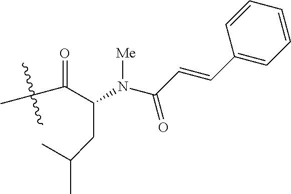 [SMILES: *C(=O)[C@@H](CC(C)C)N(C)C(=O)/C=C/c1ccccc1] |
| 72 73 74 75 | O NH O NH | CO CO —COCH(CH₃)CO— —COCH(CH₃)CO— | 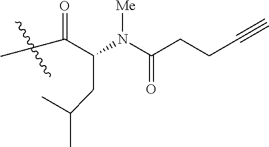 [SMILES: *C(=O)[C@@H](CC(C)C)N(C)C(=O)CCC#C] |
| 76 77 78 79 | O NH O NH | CO CO —COCH(CH₃)CO— —COCH(CH₃)CO— | 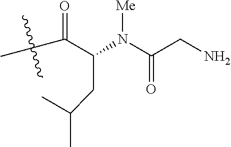 [SMILES: *C(=O)[C@@H](CC(C)C)N(C)C(=O)CN] |
| 80 81 82 83 | O NH O NH | CO CO —COCH(CH₃)CO— —COCH(CH₃)CO— | 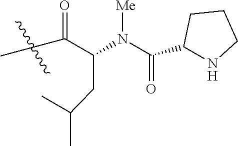 |
| 84 85 86 87 | O NH O NH | CO CO —COCH(CH₃)CO— —COCH(CH₃)CO— | 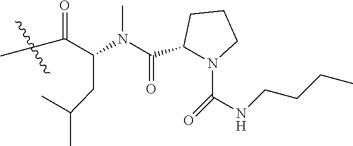 [SMILES: *C(=O)[C@@H](CC(C)C)N(C)C(=O)[C@@H]1CCCN1C(=O)NCCCC] |
| 88 89 90 91 | O NH O NH | CO CO —COCH(CH₃)CO— —COCH(CH₃)CO— | 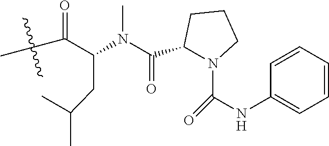 [SMILES: *C(=O)[C@@H](CC(C)C)N(C)C(=O)[C@@H]1CCCN1C(=O)Nc1ccccc1] |
| 92 93 94 95 | O NH O NH | CO CO —COCH(CH₃)CO— —COCH(CH₃)CO— | 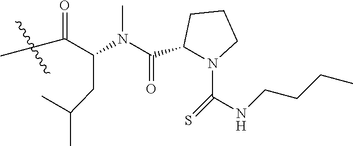 [SMILES: *C(=O)[C@@H](CC(C)C)N(C)C(=O)[C@@H]1CCCN1C(=S)NCCCC] |
| 96 97 98 99 | O NH O NH | CO CO —COCH(CH₃)CO— —COCH(CH₃)CO— | 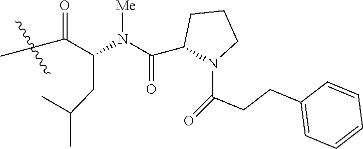 [SMILES: *C(=O)[C@@H](CC(C)C)N(C)C(=O)[C@@H]1CCCN1C(=O)CCc1ccccc1] |
| 100 101 102 103 | O NH O NH | CO CO —COCH(CH₃)CO— —COCH(CH₃)CO— | 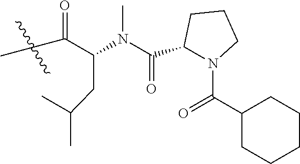 [SMILES: *C(=O)[C@@H](CC(C)C)N(C)C(=O)[C@@H]1CCCN1C(=O)C1CCCCC1] |
| 104 105 106 107 | O NH O NH | CO CO —COCH(CH₃)CO— —COCH(CH₃)CO— | 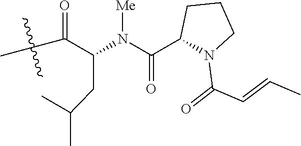 [SMILES: *C(=O)[C@@H](CC(C)C)N(C)C(=O)[C@@H]1CCCN1C(=O)/C=C/C] |
| 108 109 110 111 | O NH O NH | CO CO —COCH(CH₃)CO— —COCH(CH₃)CO— | 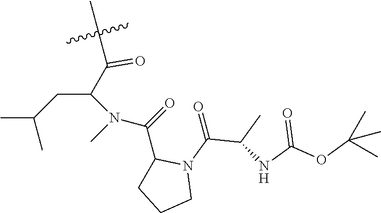 [SMILES: *C(=O)C(CC(C)C)N(C)C(=O)C1CCCN1C(=O)[C@H](C)NC(=O)OC(C)(C)C] |
| 112 113 114 115 | O NH O NH | CO CO —COCH(CH₃)CO— —COCH(CH₃)CO— | 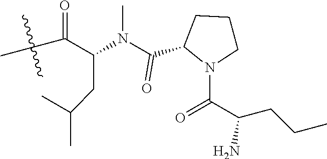 [SMILES: *C(=O)[C@@H](CC(C)C)N(C)C(=O)[C@@H]1CCCN1C(=O)[C@@H](N)CCC] |
| 116 117 118 119 | O NH O NH | CO CO —COCH(CH₃)CO— —COCH(CH₃)CO— | 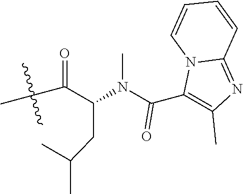 [SMILES: *C(=O)[C@@H](CC(C)C)N(C)C(=O)c1c(C)nc2ccccn12] |
| 120 121 122 123 | O NH O NH | CO CO —COCH(CH₃)CO— —COCH(CH₃)CO— | 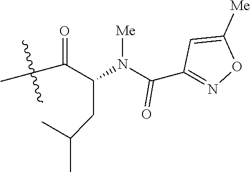 [SMILES: *C(=O)[C@@H](CC(C)C)N(C)C(=O)c1cc(C)on1] |
| 124 125 126 127 | O NH O NH | CO CO —COCH(CH₃)CO— —COCH(CH₃)CO— | 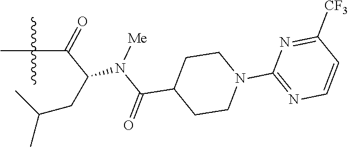 [SMILES: *C(=O)[C@@H](CC(C)C)N(C)C(=O)C1CCN(c2nccc(C(F)(F)F)n2)CC1] |
| 128 129 131 131 | O NH O NH | CO CO —COCH(CH₃)CO— —COCH(CH₃)CO— | 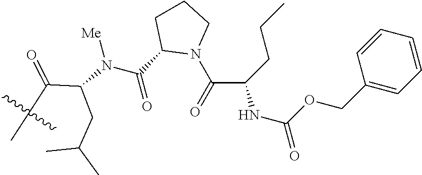 [SMILES: *C(=O)[C@@H](CC(C)C)N(C)C(=O)[C@@H]1CCCN1C(=O)[C@H](CCC)NC(=O)OCc1ccccc1] |

Following the procedures described in WO 02/02596 and in the specification, and further disclosed in the previous examples, the following compounds are obtainable:

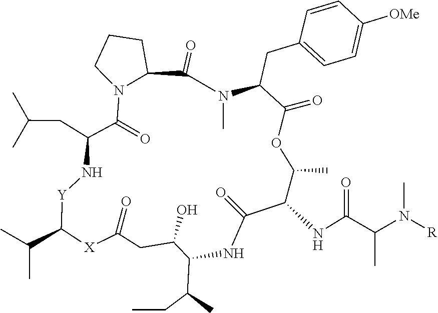

| Compound | X | Y | R |
|---|---|---|---|
| 132 133 134 135 | O NH O NH | CO CO —COCH(CH₃)CO— —COCH(CH₃)CO— | 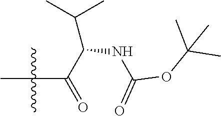 [SMILES: *C(=O)[C@@H](NC(=O)OC(C)(C)C)C(C)C] |
| 136 137 138 139 | O NH O NH | CO CO —COCH(CH₃)CO— —COCH(CH₃)CO— | 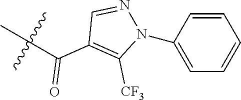 [SMILES: *C(=O)c1cnn(-c2ccccc2)c1C(F)(F)F] |
| 140 141 142 143 | O NH O NH | CO CO —COCH(CH₃)CO— —COCH(CH₃)CO— | 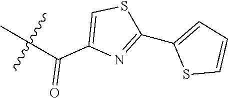 [SMILES: *C(=O)c1csc(-c2cccs2)n1] |
| 144 145 146 147 | O NH O NH | CO CO —COCH(CH₃)CO— —COCH(CH₃)CO— | 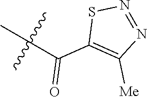 [SMILES: *C(=O)c1snnc1C] |
| 148 149 150 151 | O NH O NH | CO CO —COCH(CH₃)CO— —COCH(CH₃)CO— | 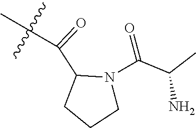 [SMILES: *C(=O)C1CCCN1C(=O)[C@H](C)N] |
| 152 153 154 155 | O NH O NH | CO CO —COCH(CH₃)CO— —COCH(CH₃)CO— | 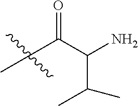 [SMILES: *C(=O)C(N)C(C)C] |
| 156 157 158 159 | O NH O NH | CO CO —COCH(CH₃)CO— —COCH(CH₃)CO— | 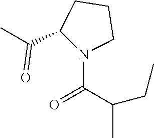 |
| 160 161 162 163 | O NH O NH | CO CO —COCH(CH₃)CO— —COCH(CH₃)CO— | 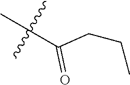 [SMILES: *C(=O)CCC] |
| 164 165 166 167 | O NH O NH | CO CO —COCH(CH₃)CO— —COCH(CH₃)CO— | 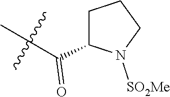 [SMILES: *C(=O)[C@@H]1CCCN1S(C)(=O)=O] |
| 168 169 170 171 | O NH O NH | CO CO —COCH(CH₃)CO— —COCH(CH₃)CO— | 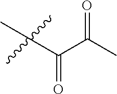 [SMILES: *C(=O)C(C)=O] |
| 172 173 174 175 | O NH O NH | CO CO —COCH(CH₃)CO— —COCH(CH₃)CO— | 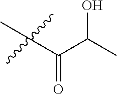 [SMILES: *C(=O)C(C)O] |
| 176 | O | CO | —SO₂Me |
| 177 | NH | CO | |
| 178 | O | —COCH(CH₃)CO— | |
| 179 | NH | —COCH(CH₃)CO— | |
| 180 181 182 183 | O NH O NH | CO CO —COCH(CH₃)CO— —COCH(CH₃)CO— | 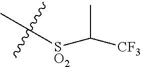 [SMILES: *S(=O)(=O)C(C)C(F)(F)F] |
| 184 185 186 187 | O NH O NH | CO CO —COCH(CH₃)CO— —COCH(CH₃)CO— | 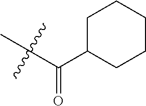 [SMILES: *C(=O)C1CCCCC1] |
| 188 189 190 191 | O NH O NH | CO CO —COCH(CH₃)CO— —COCH(CH₃)CO— | 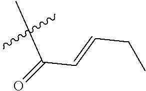 [SMILES: *C(=O)/C=C/CC] |
| 192 193 194 195 | O NH O NH | CO CO —COCH(CH₃)CO— —COCH(CH₃)CO— | 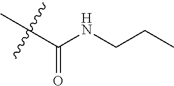 [SMILES: *C(=O)NCCC] |
| 196 197 198 199 | O NH O NH | CO CO —COCH(CH₃)CO— —COCH(CH₃)CO— | 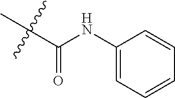 [SMILES: *C(=O)Nc1ccccc1] |
| 200 201 202 203 | O NH O NH | CO CO —COCH(CH₃)CO— —COCH(CH₃)CO— | 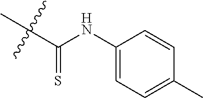 [SMILES: *C(=S)Nc1ccc(C)cc1] |
| 204 205 206 207 | O NH O NH | CO CO —COCH(CH₃)CO— —COCH(CH₃)CO— | 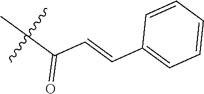 [SMILES: *C(=O)/C=C/c1ccccc1] |
| 208 209 210 211 | O NH O NH | CO CO —COCH(CH₃)CO— —COCH(CH₃)CO— | 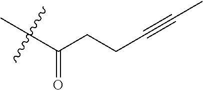 [SMILES: *C(=O)CCC#CC] |
| 212 213 214 215 | O NH O NH | CO CO —COCH(CH₃)CO— —COCH(CH₃)CO— | 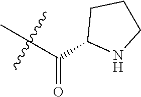 [SMILES: *C(=O)[C@@H]1CCCN1] |
| 216 217 218 219 | O NH O NH | CO CO —COCH(CH₃)CO— —COCH(CH₃)CO— | 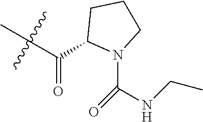 [SMILES: *C(=O)[C@@H]1CCCN1C(=O)NCC] |
| 220 221 222 223 | O NH O NH | CO CO —COCH(CH₃)CO— —COCH(CH₃)CO— | 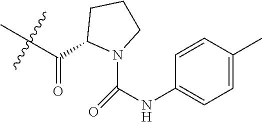 [SMILES: *C(=O)[C@@H]1CCCN1C(=O)Nc1ccc(C)cc1] |
| 224 225 226 227 | O NH O NH | CO CO —COCH(CH₃)CO— —COCH(CH₃)CO— | 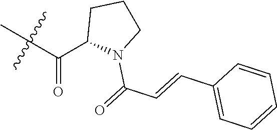 [SMILES: *C(=O)[C@@H]1CCCN1C(=O)/C=C/c1ccccc1] |
| 228 229 230 231 | O NH O NH | CO CO —COCH(CH₃)CO— —COCH(CH₃)CO— | 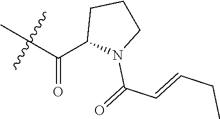 [SMILES: *C(=O)[C@@H]1CCCN1C(=O)/C=C/CC] |
| 232 233 234 235 | O NH O NH | CO CO —COCH(CH₃)CO— —COCH(CH₃)CO— | 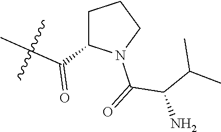 [SMILES: *C(=O)[C@@H]1CCCN1C(=O)[C@@H](N)C(C)C] |
| 236 237 238 239 | O NH O NH | CO CO —COCH(CH₃)CO— —COCH(CH₃)CO— | 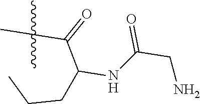 [SMILES: *C(=O)C(CCC)NC(=O)CN] |

A further compound is Compound 240, known as DidemninB and shown by the structure below:

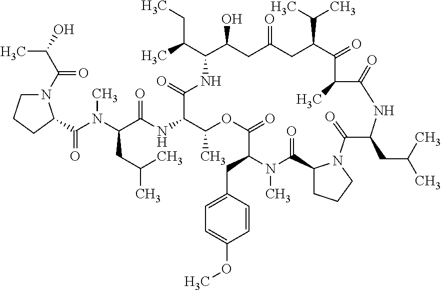 [SMILES: CC[C@H](C)[C@H]1NC(=O)[C@@H](NC(=O)[C@@H](CC(C)C)N(C)C(=O)[C@@H]2CCCN2C(=O)[C@H](C)O)[C@@H](C)OC(=O)[C@H](Cc2ccc(OC)cc2)N(C)C(=O)[C@@H]2CCCN2C(=O)[C@H](CC(C)C)NC(=O)[C@@H](C)C(=O)[C@H](C(C)C)CC(=O)C[C@@H]1O]

### Example 1

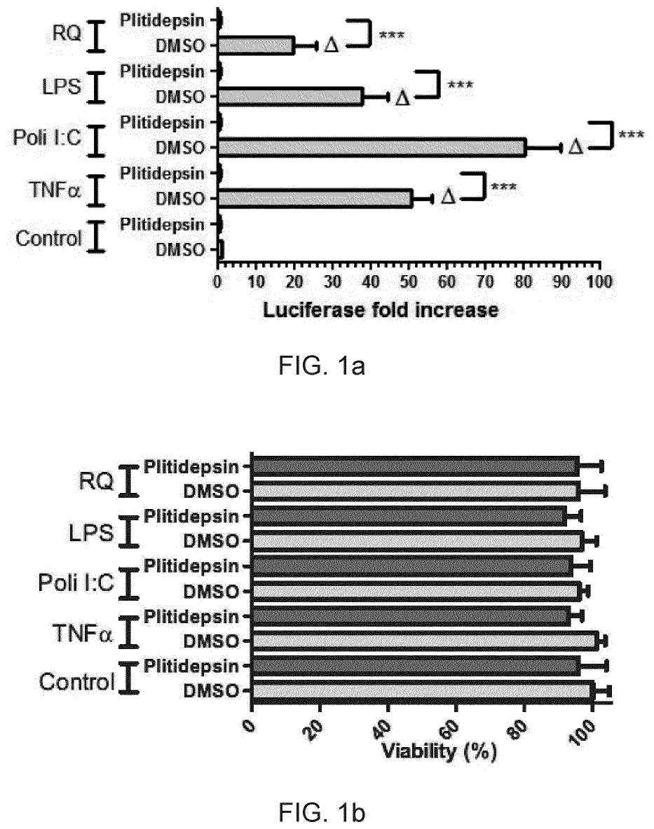

As shown in FIG. 1, PLD inhibits in vitro the transactivation of NF-κB.

We checked whether the transcriptional activity of NFκB was regulated by plitidepsin. To that end, we took advantage of THP-1 cells stably transfected with an NFκB luciferase reporter plasmid. We treated the cells with 100 ng/mL TNFα (an activator of NF-κB), 500 μg/mL poly I:C (TLR3 ligand), 10 μg/mL LPS-B5 (TLR4 ligand) or 10 μg/mL Resiquimod (TLR-7/8 ligand). The compounds were used either alone (1A grey bars) or combined with 100 nM of plitidepsin (1A black bars) for 6 hours, and quantified the luciferase activity under each condition. In the presence of each one of the TLR ligands, plitidepsin clearly inhibited the production of luciferase indicating that transactivation from NF-κB was inhibited in the presence of the drug. Survival was analysed with the MTT assay (1B grey bars (activators) and red bars (activators combined with 100 nM of plitidepsin)). No cytotoxic effect was detected.

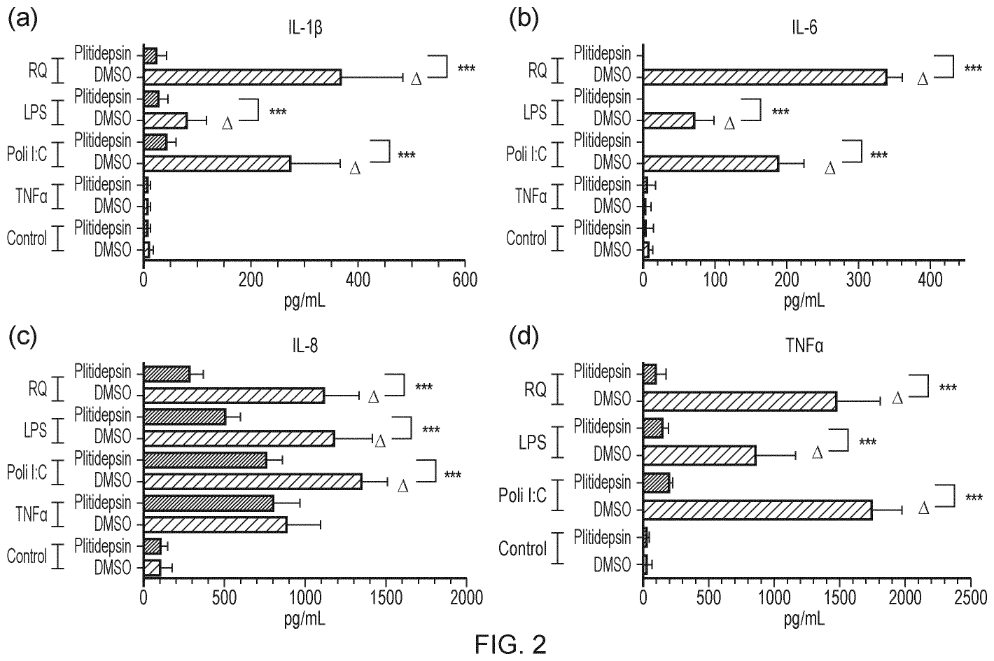

As shown in FIG. 2, PLD also inhibits in vitro the secretion of the pro-inflammatory cytokines IL-1, IL-6, IL-8 and TNF-alpha in human monocytes.

To investigate whether plitidepsin inhibits the TLR-trigged cytokine secretion, we treated THP-1 cells with 100 ng/mL TNFα (an activator of NF-κB), 500 μg/mL poly I:C (TLR3 ligand), 10 μg/mL LPS-B5 (TLR4 ligand) or 10 μg/mL Resiquimod (TLR-7/8 ligand). The compounds were used either alone (grey bars) or combined with 100 nM of plitidepsin (red bars) for 6 hours. We compared the variations in cytokine secretion in the cell culture supernatants between the different treatments by ELISA assays. As can be seen in FIG. 2, poly I:C, LPS and Resiquimod induce the secretion of IL-1, IL-6, IL-8 and TNFα. Furthermore, plitidepsin clearly inhibited the production of IL-1, IL-6, IL-8 and TNFα. TNFα failed to increase the secretion of IL-1 and IL-6. It is possible that THP-1 cells need other TNFα exposure times to secret these cytokines.

In the presence of each one of the TLR ligands, plitidepsin clearly inhibited the secretion of proinflammatory cytokines IL-1, IL-6, IL-8.

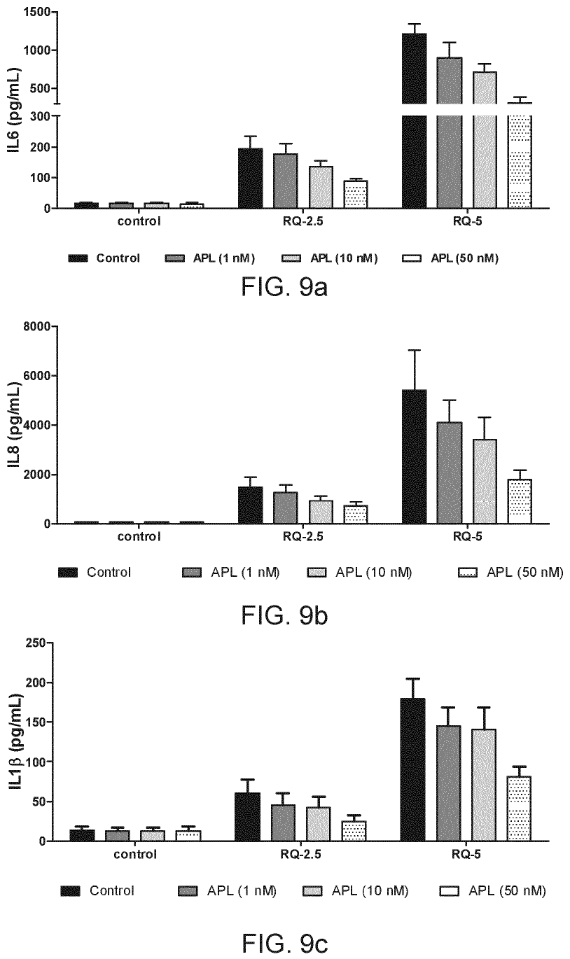

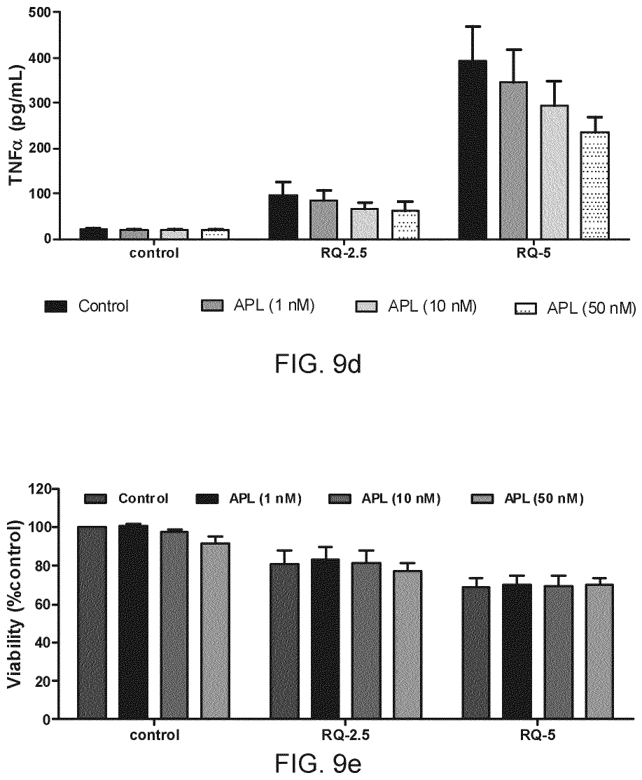

In a further in vitro experiment, the effect of plitidepsin (APL) pre-treatment on THP-1 cells was studied. Using a THP-1 NFκB luc line, 1, 10 or 50 nM of APL or DMSO (0.2%) was added 8 hours before stimulus with Resiquimod (RQ) at 2.5 or 5 μg/mL. RQ is a TLR7/8 agonist and mimics ssRNA. At 24 hours the level of cytokines or cell viability was measured. As shown in FIG. 9, PLD pre-treatment inhibited secretion of the pro-inflammatory cytokines: IL6, IL8, IL1β and TNF-α induced by RQ.

### Example 2

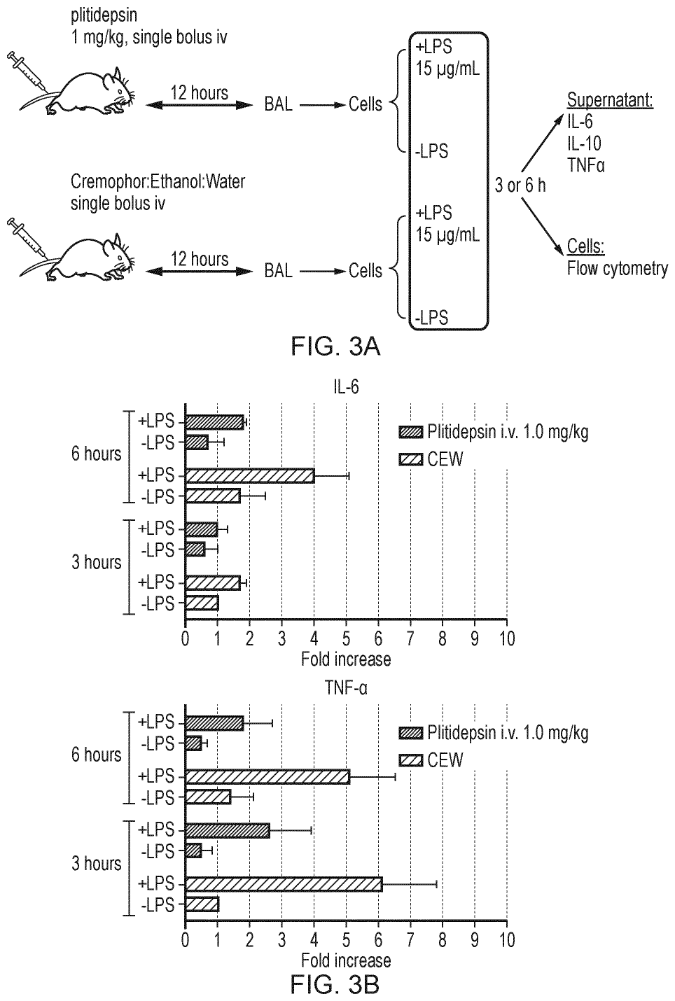

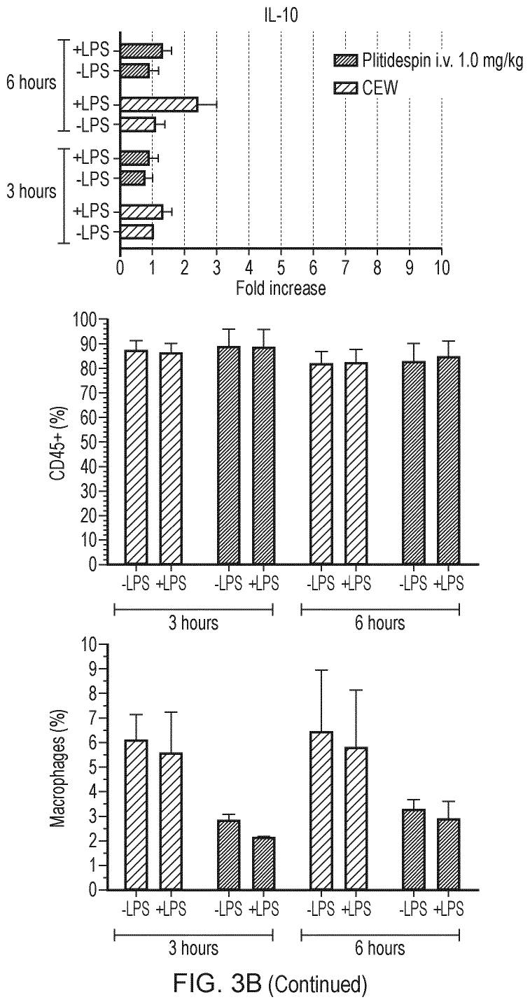

As shown in FIG. 3, PLD inhibits ex-vivo secretion of the pro-inflammatory cytokines, IL-6, IL-8 and TNF-alpha in murine isolated from BAL.

We checked whether plitidepsin inhibits the LPS-trigged cytokine secretion in alveolar macrophages. To that end mice were injected i.v. with plitidepsin (1 mg/kg) or vehicle and 12 hours after administration bronchoalveolar lavage fluid (BAL) was collected. Cells were plated and treated ex-vivo or not with 15 μg/mL of LPS-B5 for 3 or 6 hours and secreted cytokines were measured. As can be seen LPS induce the secretion of IL-6, IL-10 and TNFα (grey bars). Furthermore, in the animals treated with plitidepsin, the drug clearly inhibited the production of IL-6, and TNFα induced by LPS (red bars) and led to an overall anti-inflammatory effect.

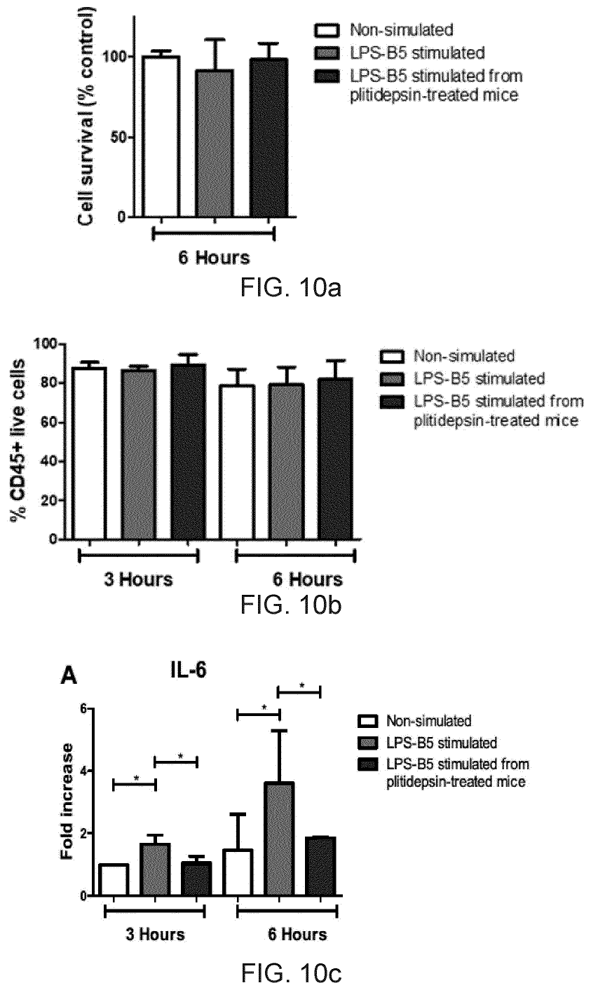

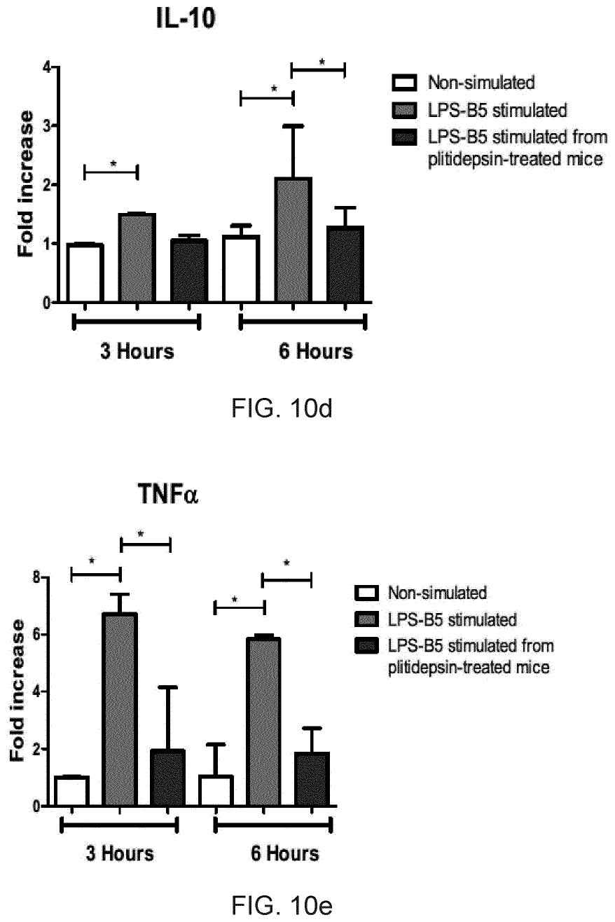

This is further shown again in FIG. 10. In the animals treated with plitidepsin, plitidepsin was able to significantly reduce the secretion of IL-6, IL-10 and TNFα induced by LPS-B5 at 3 and 6 hours in CD45⁺ cells isolated from bronco-alveolar lavages. This effect was unrelated to cell viability as shown in FIG. 10(a, b).

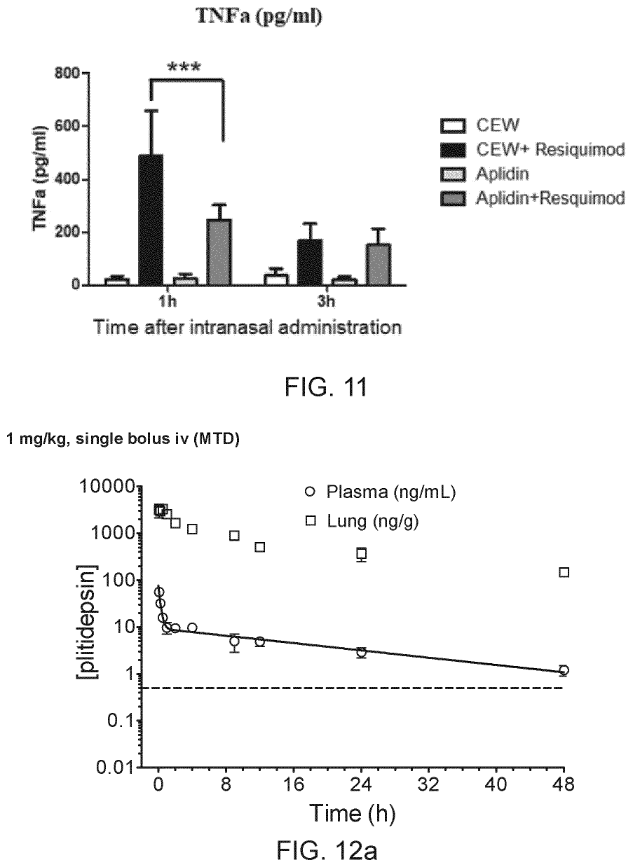

We further checked whether plitidepsin inhibits resiquimod (RQ)-trigged cytokine secretion in BALF. Mice were injected i.v. with plitidepsin (1 mg/kg) or vehicle 1 hour before a 50 μg/mouse intranasal inoculation with resiquimod. At 1 or 3 hours after intranasal administration of RQ bronchoalveolar lavage fluid (BALF) was collected. Cells were plated and secreted cytokines were measured. As can be seen in FIG. 11, RQ induces the secretion of TNFα at both 1 and 3 hours following administration. In vivo administration of PLD prevented the increased production of TNFα.

Also we check the effect of plitidepsin on alveolar macrophage recruitment. Activated monocyte-derived macrophages contribute to the COVID-19 cytokine storm by releasing massive amounts of pro-inflammatory cytokines. Bronchoalveolar lavage cells were stained and analysed by flow cytometry. Plitidepsin decrease the percentage of macrophages presents on bronchoalveolar lavage without cytotoxic effects.

### Example 3

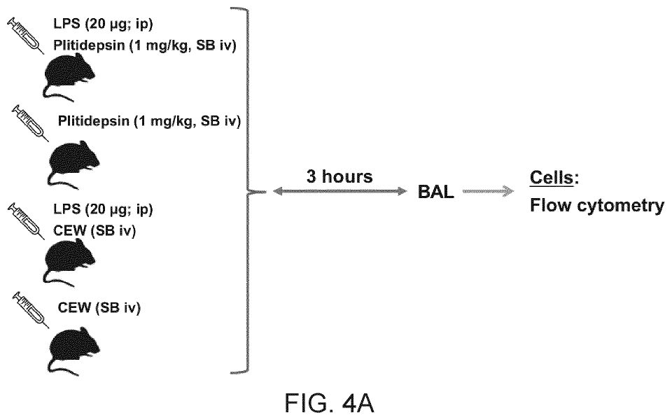

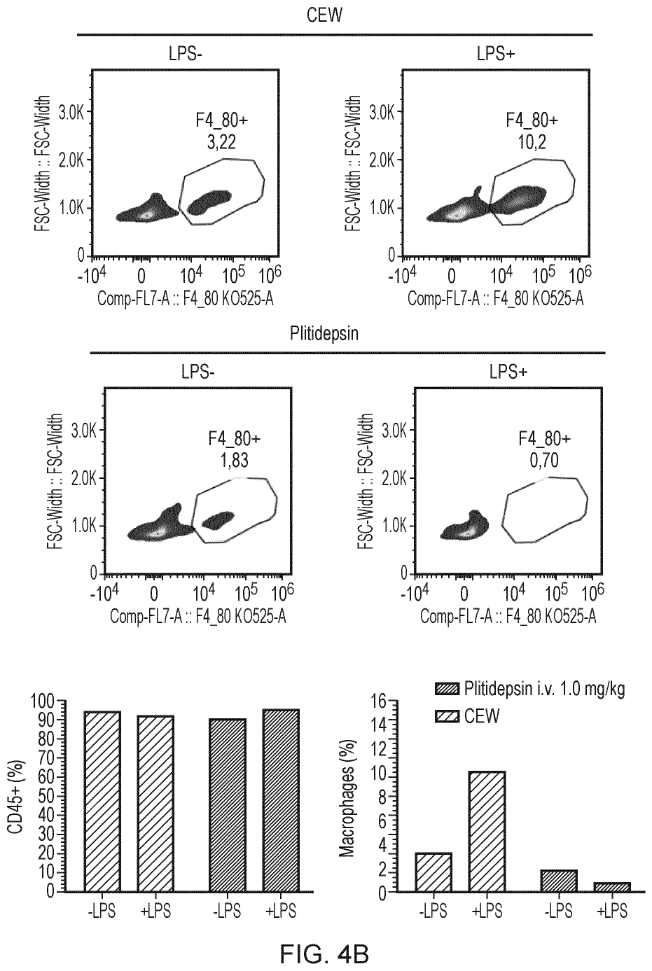

As shown in FIG. 4, after a single iv administration in mice, PLD reduces the number of macrophages in BAL.

To investigate whether plitidepsin decreases the percentage of alveolar macrophage in animals with acute inflammation, we treated mice with plitidepsin (1 mg/kg) i.v., with LPS (20 μg/kg) i.p. in sterile saline or with plitidepsin (1 mg/kg; i.v.) in combination with LPS (20 μg/kg, i.p.). Three hours later, bronchoalveolar lavages were collected. Bronchoalveolar lavage cells were obtained by centrifugation and analyzed by flow cytometry (FIG. 4b). Upper panels show the strategy of analysis of macrophage population present in the samples. Lower right panel show the same result expressed as percentage of cells. Lower left panel show the percentage of CD45+ (leucocyte marker) alive cells. As can be seen LPS induce the recruitment of alveolar macrophages. The treatment with Plitidepsin decrease the percentage of macrophages presents on bronchoalveolar lavage without cytotoxic effects.

### Example 4

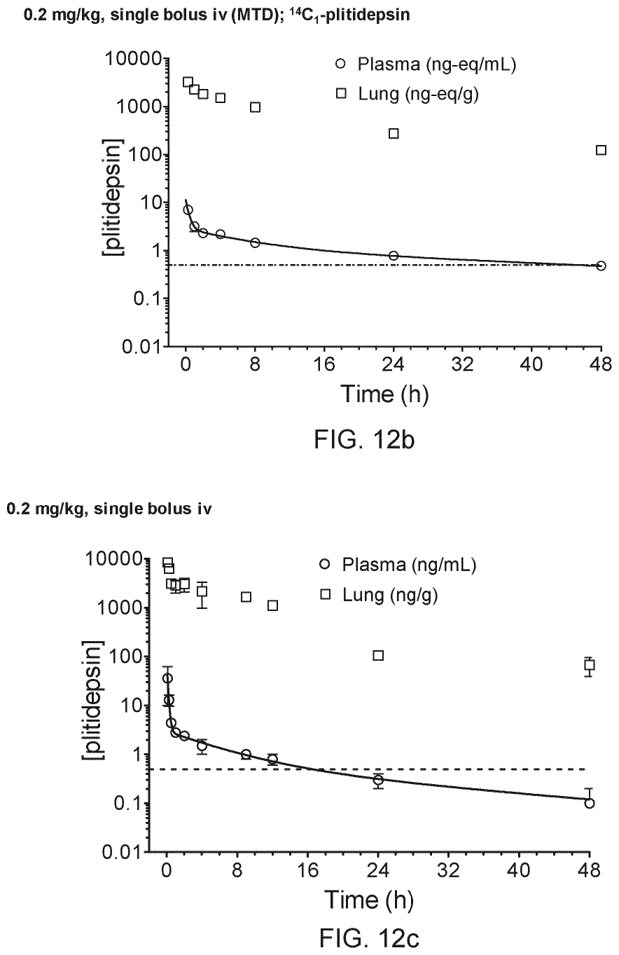

As shown in FIG. 12, PLD is distributed to the lungs in non-clinical species. In addition, similar plasma exposures are achieved in the mouse (which is the nonclinical species used in the pharmacological models) and patients.

The concentration of plitidepsin in lungs was consistently higher than that in plasma at any sampling time, with a lung-to-plasma ratio (calculated as ^{lung}AUC_{0-∞}/^{plasma}AUC_{0-∞}) in mice, rats and hamsters of 133, 460 and 909, respectively, thus confirming the distribution of plitidepsin into the lung.

TABLE 1

| Species | | Dose^{†} | Cₘₐₓ | AUC_{0-∞} | t_{1/2} | Cl | Vdₛₛ |
|---|---|---|---|---|---|---|---|
| (gender) | Strain | (mg/kg) | (ng/mL) | (ng · h/mL) | (h) | (L/h/kg) | (L7 kg) |
| Mouse (F) | C57BL6/J | 1.0^{a} | 50.7 | 225.3 | 18.2 | 4.4 | 101.8 |
| Human | — | 0.135^{b} | 29.1 | 256.0 | 20-80 | 0.7-0.9 | 29-33 |
| (M/F) | | | | | | | |
| | — | 0.02^{c} | 8.5^{d} | 174.0^{d} | — | — | — |

F, female; M, male.
^{†}Schedule:
Nonclinical species: single intravenous bolus.
Patients: 3-h intravenous infusion.
^{a}Maximum Tolerated Dose.
^{b}Calculated from the Recommended Dose of 5 mg/m2, 3-h infusion or 9.5 mg/patient.
^{c}Equivalent to 1.5 mg/patient, this being a dose used in APLICOV (1-h infusion on days 1, 2 and 3).
^{d}Estimated from plitidepsin's population PK model (CPR/2016/01), following 3 daily doses of 1.5 mg/patient.
Human body surface area, 1.9.
Human body weight, 60 kg

Materials and Methods

Transactivation Luciferase Assay.

NF-κB transactivation was assayed using the Bright-Glo™ Luciferase Assay System following the manufacturer's instructions. The NF-κB reporter (Luc)-THP-1 human monocytic, cells stably transfected with NF-κB-Luc plasmid (containing four NF-κB binding sites, a minimal promoter and a luciferase gene), were exposed to 100 ng/mL TNFα (positive control), 500 μg/mL poly (I:C) (Polyinosinic-polycytidylic), 10 μg/mL LPS-B5 (Lipopolysaccharide from Escherichia coli 055:B5) or 10 μg/mL Resiquimod. The compounds were used either alone or combined with 100 nM plitidepsin for 6 hours. Luminescence was measured in a Perkin-Elmer EnVision reader. A MTT (3-(4,5-dimethylthiazol-2-yl)-2,5-diphenyltetrazolium bromide) cell proliferation assay was simultaneously performed to control the cytotoxicity of the compounds. Cell survival was expressed as percentage of control cell growth. The data presented are the average of three independent experiments performed in triplicate.

ELISA Assays for Secreted Cytokines

THP1-NFκB-LUC cell cultures were treated as described above, and the culture medium was sampled at 6 hours post-treatment to assay for secreted cytokines by ELISA. Media samples were stored at 4-C. IL-8, IL-1β, IL-6 and TNFα protein secretion into culture medium was quantitated using highly specific and sensitive ELISA kits. Human IL-lb, human IL-6, human IL-8 and human TNF OptEIA™ ELISA kits were obtained from BD Biosciences and performed as described by the manufacturer. The data presented are the average of three independent experiments performed in triplicate.

MTT Assay

Cells were seeded in 96 well microtiter plates and allowed to stand for 24 hours at 37° C. and 5% CO₂ before treatment described above. After 6 hours of continuous treatment, cellular viability was estimated from conversion of MTT to its coloured reaction product, MTT formazan, which was dissolved to measure its absorbance at 540 nm. Data presented here are representative from a series of at three independent experiments performed in triplicate.

“In Vivo” and “Ex Vivo” Treatments.

Mice were randomized into groups of five animals to receive the treatments. Mice were injected intra venous (i.v.) with plitidepsin (1 mg/kg) and 12 hours after administration were euthanized. Control group received plitidepsin vehicle diluted with saline (Cremophor/Ethanol/Water). Bronchoalveolar lavage fluid (BAL) of each group was collected and centrifugated to obtain bronchoalveolar lavage cells. Cells underwent red blood cell lysis (Roche) and were plated and treated ex-vivo or not with 15 μg/mL of LPS-B5 for 3 or 6 hours. Secreted cytokines were measured using highly specific and sensitive ELISA kits. Mouse IL-6, mouse IL-10 and mouse TNF DuoSet ELISA kits were obtained from R&D Systems and performed as described by the manufacturer. Data presented here are representative from a series of at three independent experiments.

Animal Inflammation Model.

Mice were randomized into groups of two animals to receive the treatments. Mice were challenged with plitidepsin (1 mg/kg) intra venous (i.v.), with LPS (20 μg/kg) intra peritoneal (i.p.) in sterile saline or with plitidepsin (1 mg/kg; i.v.) in combination with LPS (20 μg/kg, i.p.). The control group received plitidepsin vehicle (Cremophor/Ethanol/Water) diluted with saline. Three hours later, animals were euthanised and bronchoalveolar lavage collected (a total of 1.2 ml, PBS). Bronchoalveolar lavage cells were obtained by centrifugation and analyzed by flow cytometry. Data presented here are representative from a series of at three independent experiments.

In another inflammation model, mice were randomized into groups of two animals to receive the treatments. Mice were challenged with plitidepsin (1 mg/kg) intra venous (i.v.) followed by Resiquimod (50 μg/mouse, intranasal) 1 hour latter. The control group received plitidepsin vehicle (Cremophor/Ethanol/Water) diluted with saline. One and 3 hours later, animals were euthanized, bronchoalveolar lavage collected (a total of 1.2 ml, PBS) and then, TNFα quantified by ELISA kits. Data presented here are representative from a series of at three independent experiments.

Analysis of Macrophages by Flow Cytometry.

Bronchoalveolar lavage cells were stained with anti-F4/80-BV510, CD45-APC700, CD11b-BV650, CD11c-APC-Fire, CD24-PC7 and Ly6C-BV605 monoclonal antibodies (Biolegend) and a LIVE/DEAD™ Fixable Green Dead Cell Stain Kit, for 488 nm excitation (Thermofisher). Macrophages (F4/80+) were gated on alive immune cells (CD45+ LIVE/DEAD dye−), while alveolar macrophages (F4/80+ CD24−) were specifically gated on CD11c+CD11b− population from alive immune cells. Isotype controls and compensation beads were used to set compensations and gating strategies.

### Example 5

A multicenter, randomized, parallel and proof of concept study was undertaken to evaluate the safety profile of three doses of Plitidepsin in patients with COVID-19 requiring hospitalization. Study details are available through ClinicalTrials.gov Identifier: NCT04382066.

Patients included in the study were randomised in a 1:1:1 ratio to receive:
Arm A) 1.5 mg of plitidepsin administered as a 1.5-hour infusion, once a day for 3 consecutive days (total dose 4.5 mg).
Arm B) 2.0 mg of plitidepsin administered as a 1.5-hour infusion, once a day for 3 consecutive days (total dose 6.0 mg).
Arm C) 2.5 mg of plitidepsin administered as a 1.5-hour infusion, once a day for 3 consecutive days (total dose 7.5 mg).

All patients received the following prophylactic medications 20-30 minutes before the infusion of plitidepsin:
Diphenhydramine hydrochloride 25 mg iv or equivalent.
Ranitidine 50 mg iv or equivalent.
Dexamethasone 6.6 mg intravenous.
Ondansetron 8 mg i.v. in slow infusion of 15 minutes or equivalent.

Patients included in the study will receive treatment for 3 days.

Plitidepsin is supplied as a powder for concentrate for solution for infusion at a concentration of 2 mg/vial. Before use, the vials are reconstituted with 4 ml of reconstitution solution to obtain a colourless to slightly yellowish solution containing 0.5 mg/ml of plitidepsin, 25 mg/ml of mannitol, 0.15 ml/ml of macrogolglycerol ricinoleate oil, 0.15 ml/ml of ethanol and 0.70 ml/ml of water for injection. An additional dilution should be made in any suitable intravenous solution prior to infusion.

Plitidepsin 2 mg is supplied in a Type I clear glass vial with a bromobutyl rubber stopper covered with an aluminium seal. Each vial contains 2 mg of plitidepsin.

The solvent for the reconstitution of macrogolglycerol ricinoleate (polyoxyl 35 castor oil)/absolute ethanol/water for injection, 15%/15%/70% (v/v/v) is supplied in a Type I colourless glass vial. The ampoules have a volume of 4 ml.

Plitidepsin will be labelled with the study protocol code, the batch number, the content, the expiry date, the storage conditions, the name of the investigator and the sponsor. The study drug will be labelled in accordance with Annex 13 of the European Good Manufacturing Practices. Plitidepsin should be stored between 2° C. and 8° C. and the vials should be kept in the outer carton to protect them from light. The drug in these conditions is stable for 60 months.

After reconstitution of the 2 mg plitidepsin vial with 4 ml of the solution for reconstitution of macrogolglycerol ricinoleate/ethanol/water for injection, the reconstituted solution should be diluted and used immediately after preparation. If not used immediately, storage times and conditions until use are the responsibility of the user. The reconstituted concentrated solution of the drug product has been shown to be physically, chemically and microbiologically stable for 24 hours under refrigerated conditions (5° C.±3° C.) and for 6 hours when stored in the original vial under indoor light at room temperature. If storage is required before administration, solutions should be stored refrigerated and protected from light and should be used within 24 hours after reconstitution.

Interim Results

Patient 1-50 year old male, bilateral pneumonia. Received PLD 1.5 mg×3. PCR COVID 19 test: POSITIVE at baseline, converted to NEGATIVE (no viral load) by day 4. Acute clinical improvement. Hospital discharge by day 7. PLD achieved an acute clinical improvement, including removing all viral burden and treating bilateral pneumonia to enable hospital discharge by day 7.

Patient 2: 40 year old male, bilateral pneumonia. Received PLD 1.5 mg×3. By day six, lack of improvement and cross over to Remdesivir+TOL+Corticoids+Opiates. PCR converted to negative by day 15, Hospital discharge by Day 19.

Patient 3: 53 year old male, bilateral pneumonia. Received PLD 1.5 mg×3. PLD prevented clinical deterioration. Hospital discharge by day 10, PCR converted to negative by day 31.

Patient 4: 42 year old male, bilateral pneumonia. Received PLD 2.0 mg×3. Corticoid therapy required. PCR COVID 19 test: POSITIVE at baseline, and still positive at day 7. By day 15 the patient was PCR negative. Patient recovered sufficiently for hospital discharge by day 10.

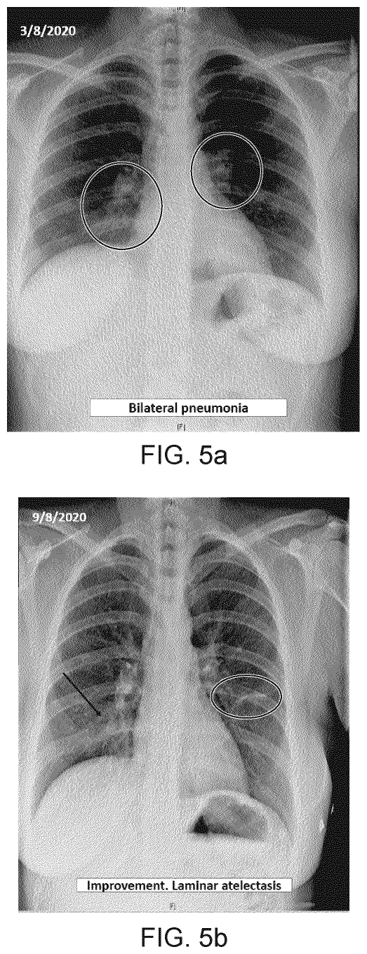

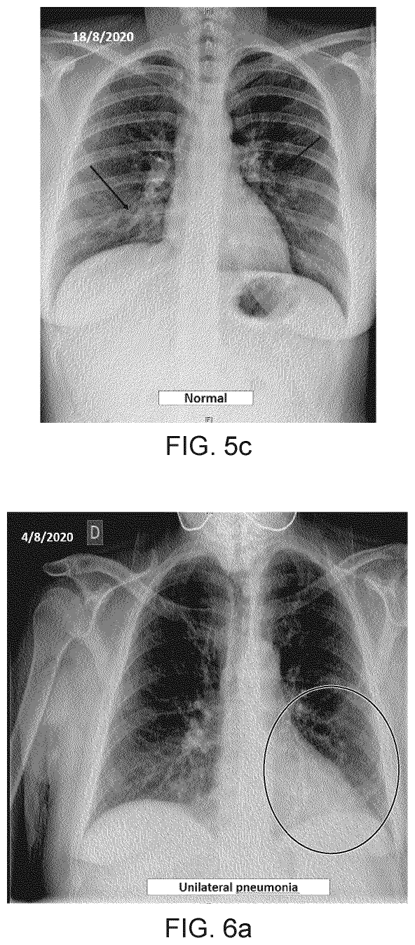

Patient 5: 33 year old female, bilateral pneumonia at entry. Received PLD 1.5 mg×3. PCR COVID 19 test: POSITIVE at baseline, converted to NEGATIVE (no viral load) by day 4. Bilateral pneumonia resolved by day 6 (normal Rx Lung). Major clinical improvement. Hospital discharge by day 8. X-rays showing pneumonia resolution shown in FIG. 5a-c. Bilateral pneumonia is evident in FIG. 5a. After treatment with PLD, improvement was seen on day 6. Laminar atelectasis is evidenced FIG. 5b. A follow up x-ray on day 15 showed return to normal FIG. 5c. PLD 1.5 mg×3 removed viral load by day 4. PLD achieved major clinical improvement, including removing all viral burden and treating bilateral pneumonia to enable hospital discharge by day 8.

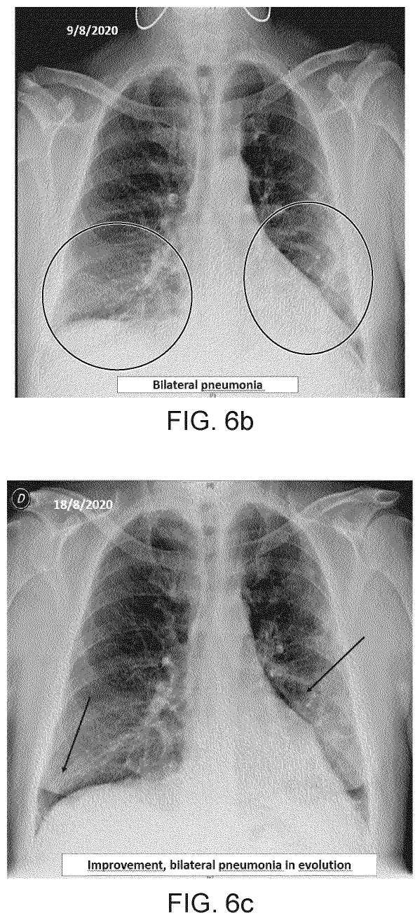

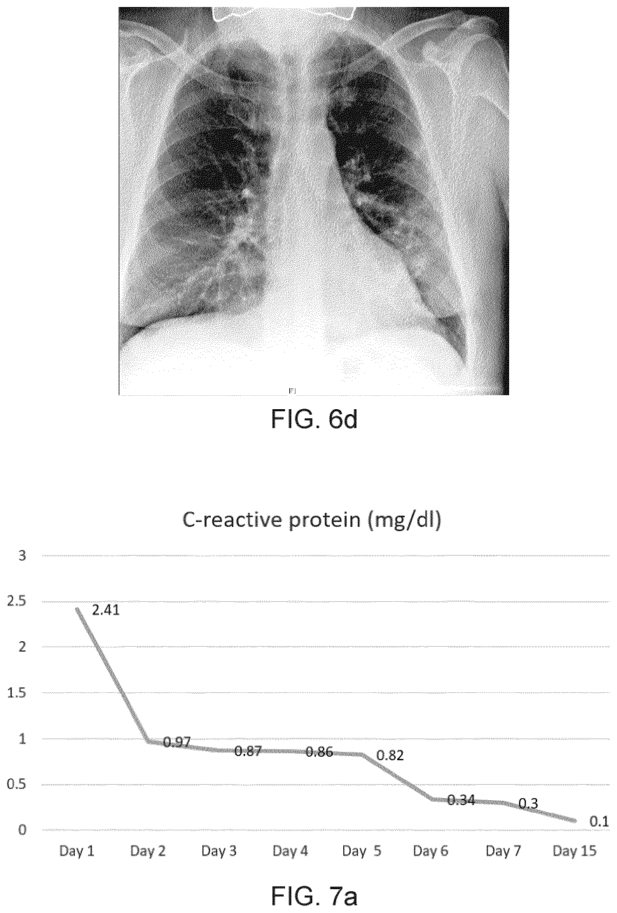

Patient 6: 69 year old female, highly symptomatic COPD. Unilateral pneumonia on entry. Received PLD 1.5 mg×3. PCR COVID 19 test: POSITIVE at baseline, converted to NEGATIVE (no viral load) by day 7 as shown. Major clinical improvement seen. Patient discharged by day 8. X-rays showing pneumonia progression shown in FIG. 6a-c. Unilateral pneumonia is evident in FIG. 6a which progressed to bilateral pneumonia in FIG. 6b. In FIG. 6c, improvement is seen. PLD achieved major clinical improvement, including removing all viral burden and treating pneumonia as shown in FIG. 6d to enable hospital discharge by day 8.

Patient 7: 39 year old female, pulmonary infiltrates. Received PLD 2.0 mg×3. PCR COVID 19 test: POSITIVE at baseline, converted to NEGATIVE (no viral load) by day 7. Following treatment with PLD, major clinical improvement. Hospital discharge by day 8.

Patient 8: 32 year old male. Received PLD 1.5 mg×3. Not evaluable for efficacy, hospital discharge by day 4.

Patient 9: 34 year old male. Received PLD 2.0 mg×3. PCR COVID 19 test: POSITIVE at baseline and still positive at day 7. However, major clinical improvement and hospital discharge by day 8.

C-Reactive Protein Tests

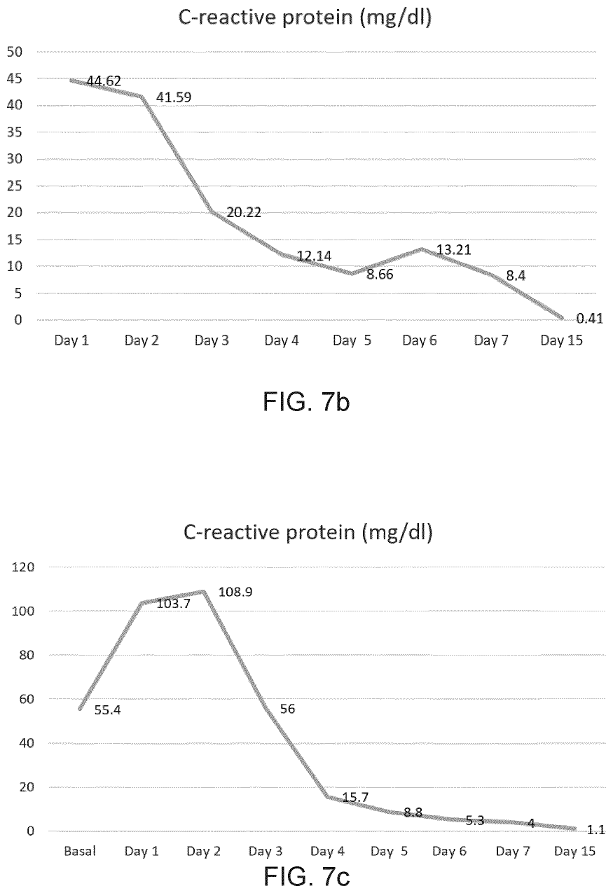

The effect of PLD on inflammatory cytokines was also measured for patients 5, 7 and 9 and the results of C-reactive protein tests are shown in FIG. 7. With patient 5 (FIG. 7a), following administration of PLD, an acute fall is seen by day 2. With patients 7 (FIG. 7b) and 9 (FIG. 7c), following administration of PLD, an acute fall is seen by day 3. These data demonstrate anti-inflammatory properties of PLD.

Overall, upon completion of the study, 45 patients hospitalised for COVID 19 were randomised to treatment with plitidepsin at doses of 1.5, 2.0, and 2.5 mg daily for 3 days. Treatment was well tolerated in all 3 dose cohorts. Treatment outcomes, assessed by hospital discharge rate, were driven by disease severity and viral load at baseline. Across dose cohorts, 100% (9/9) patients with mild disease, 82% (23/28) with moderate disease, and 57% (4/7) with severe disease were discharged by Day 15.

### Example 6

The aim here was to evaluate in vivo the effects of plitidepsin in the treatment of severe pneumonia caused by the mouse-adapted A/H1N1 influenza virus infection (A/Puerto Rico/8/34).

Experimental set-up: To achieve this objective we employed an in vivo model of viral pathogenesis based on the administration of high-dose of PR8 influenza virus (2×105 pfu), which generated a severe infection in the lungs. We then evaluated the therapeutic effect of plitidepsin on severe influenza virus infection in mice. Female mice at the age of 9 weeks were anesthetized by intraperitoneal injection of ketamine-xylazine solution and infection was performed by intranasal administration of virus solution PBS into 20 ul per nares.

Mice that were receiving the treatment were injected subcutaneously with 0.3 mg/kg or 0.15 mg/kg of plitidepsin. Subsequently, survival and body weight loss was monitored until day 3 p.i. No death mice or mice with a weight loss of more than 30% of the starting body weight was recorded during the time of the treatment.

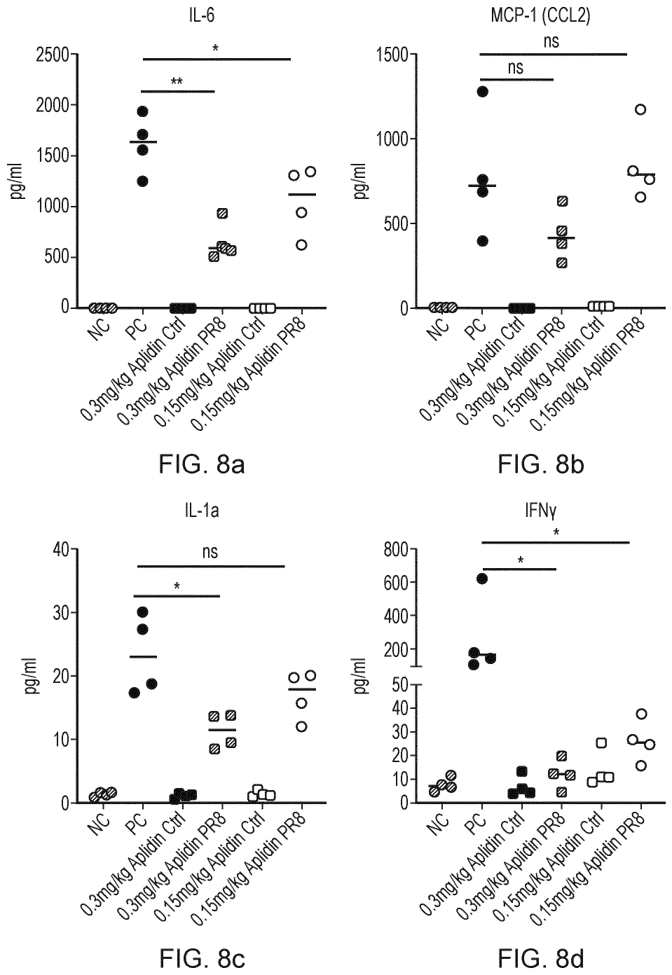

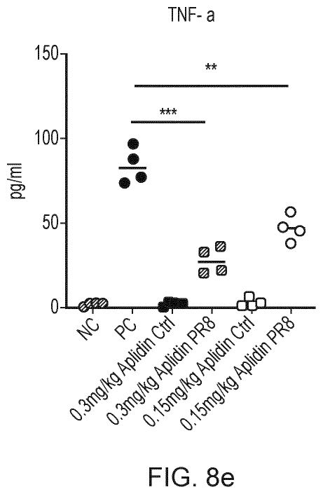

The control of influenza infection in the airways is mediated by enhanced inflammation in the bronchoalveolar lavage fluid (BALF). FIG. 8 shows the inflammatory profile in the BALF of infected mice with or without treatment with plitidepsin. Among the major pro-inflammatory cytokines, plitidepsin strongly reduced the levels of IL-6 (FIG. 8a), CCL2 (FIG. 8b), IL-la (FIG. 8c), IFN-γ (FIG. 8d) and TNF-α (FIG. 8e). Mice that were receiving only half-dose of the drug were less protected and showed an intermediated phenotype.

The BALF cellular composition is defined as a marker of lung immune response viral infection. Quantitative measurement of infiltrating cells in correlation to inflammatory cytokine levels was assessed in influenza infected mice. Treatment with plitidepsin did not reduce the total cellular composition of the BALF (CD45⁺×10⁶).

All together, these results confirmed that three subsequent administrations of (total dose of 0.9 mg/kg) of plitidepsin in influenza infected mice can positively reduce inflammation, as shown by the reduction of the early pro-inflammatory cytokines by the treatment.

### REFERENCES

Racke M K, Drew P D. Toll-like receptors in multiple sclerosis. Curr Top Microbiol Immunol. 2009; 336:155-168. doi:10.1007/978-3-642-00549-7_9
Elshabrawy H A, Essani A E, Szekanecz Z, Fox D A, Shahrara S. TLRs, future potential therapeutic targets for RA. Autoimmun Rev. 2017; 16(2):103-113. doi:10.1016/j.autrev.2016.12.003
Mohammad Hosseini A, Majidi J, Baradaran B, Yousefi M. Toll-Like Receptors in the Pathogenesis of Autoimmune Diseases. Adv Pharm Bull. 2015; 5(Suppl 1):605-614. doi:10.15171/apb.2015.082
Huang Q Q, Pope R M. The role of toll-like receptors in rheumatoid arthritis. Curr Rheumatol Rep. 2009; 11(5):357-364. doi:10.1007/s11926-009-0051-z
Marshak-Rothstein A. Toll-like receptors in systemic autoimmune disease. Nat Rev Immunol. 2006; 6(11):823835. doi:10.1038/nri1957
Prinz M, Garbe F, Schmidt H, Mildner A, Gutcher I, Wolter K, Piesche M, Schroers R, Weiss E, Kirschning C, Rochford C, Bruck W, Becher B. Innate immunity mediated by TLR9 modulates pathogenicity in an animal model of multiple sclerosis. J Clin Invest. 2006; 116(2):456-464. doi:10.1172/JCI26078
O'Reilly S. Toll Like Receptors in systemic sclerosis: An emerging target. Immunology Letters. 2018; 195:2-8. doi:10.1016/j.imlet.2017.09.001
Kiripolsky J, Kramer J. Current and Emerging Evidence for Toll-Like Receptor Activation in Sjögren's Syndrome. J Immunol Res. 2018; 1246818. doi:10.1155/2018/1246818 Feng Y, Chao W. Toll-Like Receptors and Myocardial Inflammation. Int J Inflam. 2011; 170352. doi:10.4061/2011/170352
Zipris D. Toll-like receptors and type 1 diabetes. Adv Exp Med Biol. 2010; 654:585-610. doi:10.1007/978-90-481-3271-3_25
Falck-Hansen M, Kassiteridi C, Monaco C. Toll-Like Receptors in Atherosclerosis. Int J Mol Sci. 2013; 14(7):14008-14023. doi:10.3390/ijms140714008

1. A compound of general formula I 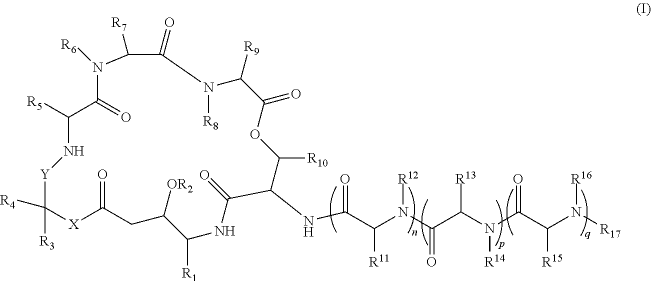
wherein X is selected from O and NH;
Y is selected from CO and —COCH(CH₃)CO—;
each n and p is independently selected from 0 and 1, and q is selected from 0, 1 and 2;
each R₁, R₃, R₅, R₉, R₁₁, and R₁₅ is independently selected from hydrogen, substituted or unsubstituted C₁-C₆ alkyl, substituted or unsubstituted C₂-C₆ alkenyl, and substituted or unsubstituted C₂-C₆ alkynyl;
R₂ is selected from hydrogen, CORₐ, COORₐ, substituted or unsubstituted C₁-C₆ alkyl, substituted or unsubstituted C₂-C₆ alkenyl, and substituted or unsubstituted C₂-C₆ alkynyl;
each R₄, R₈, R₁₀, R₁₂, and R₁₆ is independently selected from hydrogen and substituted or unsubstituted C₁-C₆ alkyl;
each R₇ and R₁₃ is independently selected from hydrogen, substituted or unsubstituted C₁-C₆ alkyl, substituted or unsubstituted C₂-C₆ alkenyl, and substituted or unsubstituted C₂-C₆ alkynyl; each R₆ and R₁₄ is independently selected from hydrogen and substituted or unsubstituted C₁-C₆ alkyl; or R₆ and R₇ and/or R₁₃ and R₁₄ together with the corresponding N atom and C atom to which they are attached may form a substituted or unsubstituted heterocyclic group;
R₁₇ is selected from hydrogen, CORₐ, COORₐ, CONHR_{b}, COSR_{c}, (C═NR_{b})ORₐ, (C═NR_{b})NHR_{b}, (C═NR_{b})SR_{c}, (C═S)ORₐ, (C═S)NHR_{b}, (C═S)SR_{c}, SO₂R_{c}, SO₃R_{c}, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, with the proviso that when n, p, and q are 0 then R₁₇ is not hydrogen; and
each Rₐ, R_{b}, and R_{c} is independently selected from hydrogen, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group;
or a pharmaceutically acceptable salt or stereoisomer thereof, for use in the treatment of an autoimmune condition.

2. A compound of claim 1, wherein the autoimmune condition is caused by the activation of one or more Toll-like receptor (TLR).

3. A compound of claim 1, wherein the autoimmune condition is characterised by increased signalling through at least one or more Toll-like receptor (TLR).

4. A compound of claim 1, wherein the autoimmune condition is characterised by increased levels of at least one pro-inflammatory cytokines.

5. A compound of any of claims 1 to 4, wherein the autoimmune condition is selected from systemic lupus erythematosus (SLE), rheumatoid arthritis (RA), multiple sclerosis (MS), scleroderma, Sjögren's syndrome, autoimmune myocarditis, type 1 diabetes, and atherosclerosis.

6. A compound of claim 5, wherein the autoimmune condition is RA.

7. A compound according to any one of claims 1 to 6, wherein R₃ and R₄ are independently selected from hydrogen and substituted or unsubstituted C₁-C₆ alkyl; preferably wherein R₃ is isopropyl and R₄ is hydrogen.

8. A compound according to any one of claims 1 to 7, of general formula II, wherein R₃ and R₄ are methyl.

9. A compound according to any preceding claim, wherein R₁₁ is selected from hydrogen and substituted or unsubstituted C₁-C₆ alkyl; preferably wherein R₁₁ is methyl or isobutyl.

10. A compound according to any preceding claim, of general formula III wherein R₁₁ is methyl and n=1.

11. A compound according to any preceding claim, wherein R₁, R₅, R₉, and R₁₅ are independently selected from hydrogen and substituted or unsubstituted C₁-C₆ alkyl; preferably wherein R₁ is selected from sec-butyl and isopropyl, R₅ is isobutyl, R₉ is p-methoxybenzyl, and R₁₅ is selected from methyl and benzyl.

12. A compound according to any preceding claim, wherein R₈, R₁₀, R₁₂, and R₁₆ are independently selected from hydrogen and substituted or unsubstituted C₁-C₆ alkyl; preferably wherein R₈, R₁₀ and R₁₂ are methyl, and R₁₆ is hydrogen.

13. A compound according to any preceding claim, wherein R₆ and R₁₄ are independently selected from hydrogen and substituted or unsubstituted C₁-C₆ alkyl; preferably wherein R₆ is selected from hydrogen and methyl, and R₁₄ is hydrogen.

14. A compound according to any preceding claim, wherein R₇ and R₁₃ are independently selected from hydrogen and substituted or unsubstituted C₁-C₆ alkyl; preferably wherein R₇ is methyl and R₁₃ is selected from hydrogen, methyl, isopropyl, isobutyl, and 3-amino-3-oxopropyl.

15. A compound according to any of claims 1 to 12, wherein R₆ and R₇ and/or R₁₃ and R₁₄ together with the corresponding N atom and C atom to which they are attached form a substituted or unsubstituted pyrrolidine group.

16. A compound according to any preceding claim, wherein R₂ is selected from hydrogen, substituted or unsubstituted C₁-C₆ alkyl, and CORₐ, and wherein Rₐ is a substituted or unsubstituted C₁-C₆ alkyl; preferably wherein R₂ is hydrogen.

17. A compound according to any preceding claim, wherein R₁₇ is selected from hydrogen, CORₐ, COORₐ, CONHR_{b}, (C═S)NHR_{b}, and SO₂R_{c}, and wherein each Rₐ, R_{b}, and R_{c} is independently selected from substituted or unsubstituted C₁-C₆ alkyl, substituted or unsubstituted C₂-C₆ alkenyl, substituted or unsubstituted C₂-C₆ alkynyl, substituted or unsubstituted aryl, and substituted or unsubstituted heterocyclic group; preferably wherein R₁₇ is selected from hydrogen, COObenzyl, CObenzo[b]thiophen-2-yl, SO₂(p-methylphenyl), COCOCH₃ and COOC(CH₃)₃.

18. A compound according to any preceding claim, wherein X is NH.

19. A compound according to any of claims 1 to 17, wherein X is O.

20. A compound according to any preceding claim wherein Y is CO.

21. A compound according to any of claims 1 to 19, wherein Y is —COCH(CH₃)CO—.

22. A compound according to any one of claims 1 to 6, having the following structure: 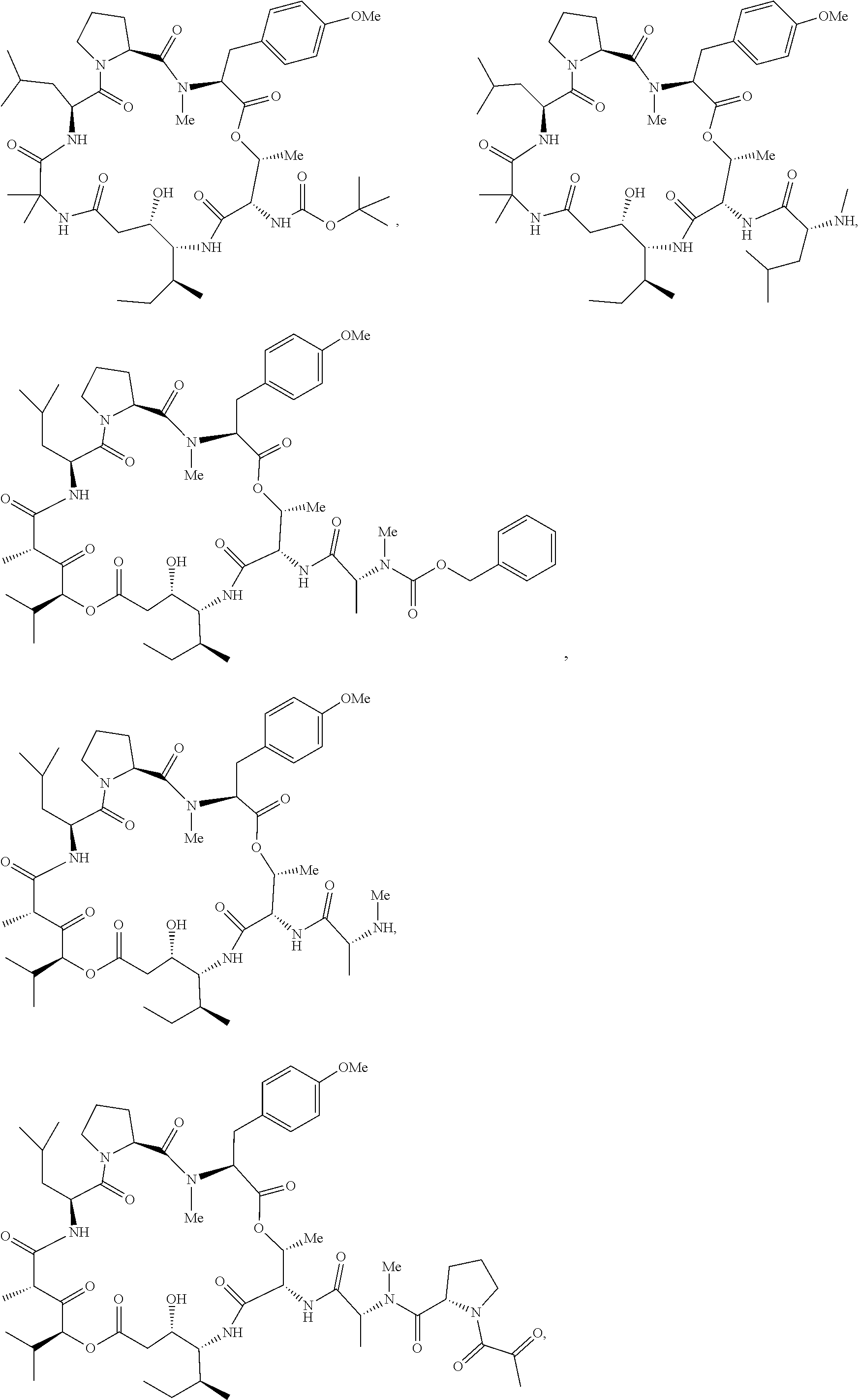 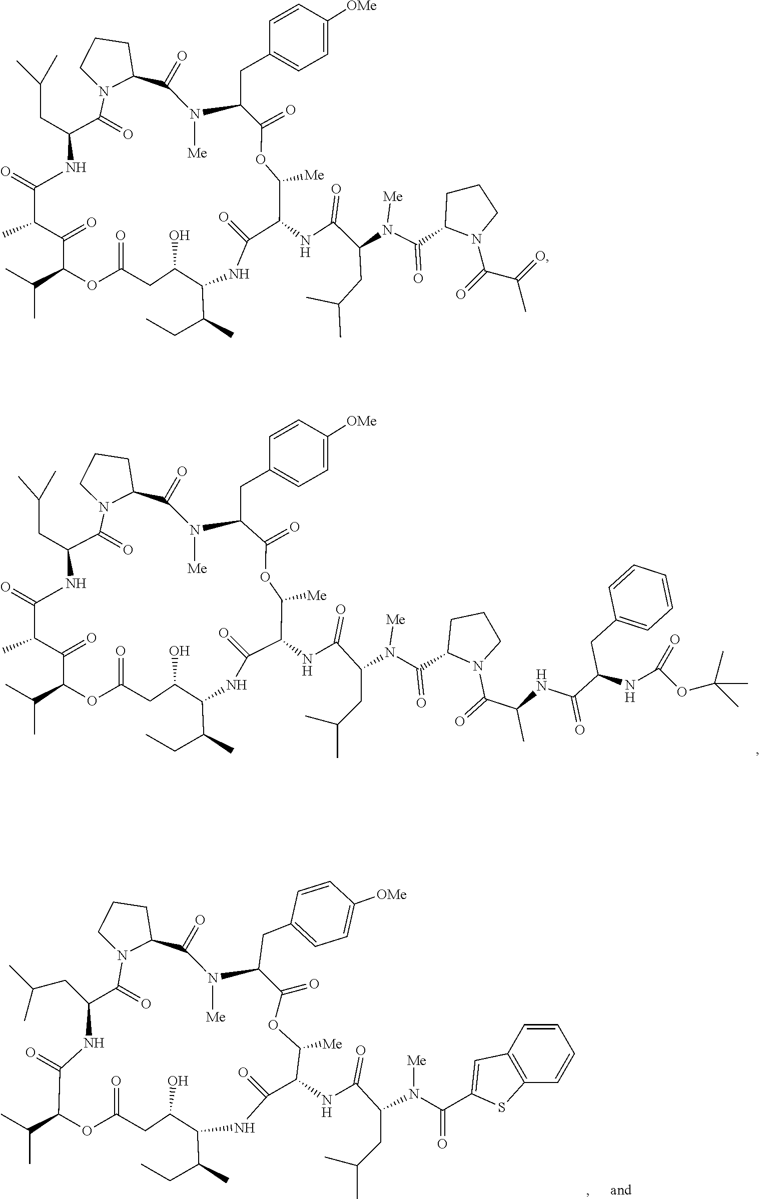 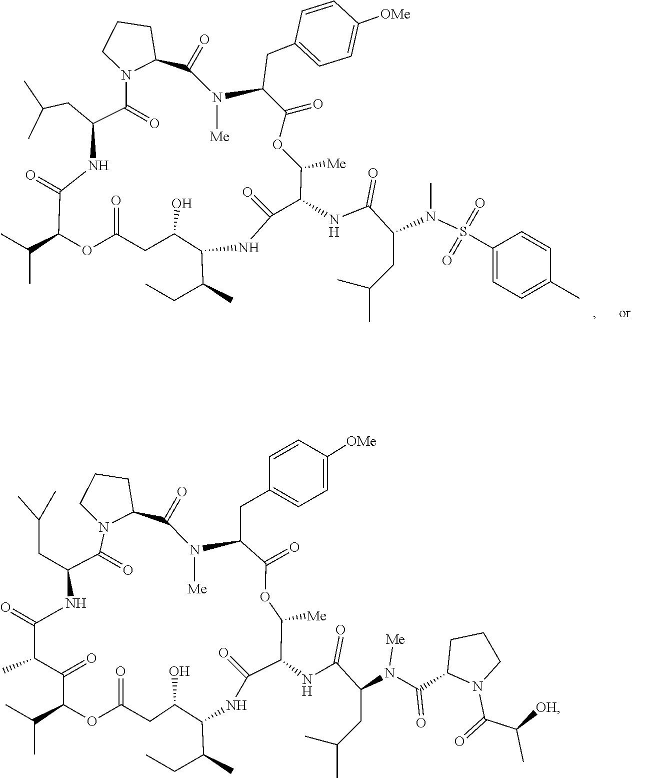
or pharmaceutically acceptable salts or stereoisomers thereof.

23. A compound according to any one of claims 1 to 6, wherein the compound is PLD, or pharmaceutically acceptable salts or stereoisomers thereof.

24. A compound according to any one of claims 1 to 6, wherein the compound is didemninB, or pharmaceutically acceptable salts or stereoisomers thereof.

25. A compound according to any one of claims 1 to 6, wherein the compound is PLD.

26. A compound according to any one of claims 1 to 6, wherein the compound is Didemnin B.

27. A pharmaceutical composition comprising a compound as defined in any of claims 1 to 26, or a pharmaceutically acceptable salt or stereoisomer thereof, and a pharmaceutically acceptable carrier, for use in the treatment of an autoimmune condition.

28. Use of a compound as defined in to any of claims 1 to 26, or a pharmaceutically acceptable salt or stereoisomer thereof, in the manufacture of a medicament for the treatment of an autoimmune condition.

29. A method of treating an autoimmune condition, wherein the method comprises administering to an individual in need thereof, a therapeutically effective amount of a compound as defined in any of claims 1 to 26, or a pharmaceutically acceptable salt or stereoisomer thereof.

30. A kit comprising the compound as defined in any of claims 1 to 26, together with instructions for treating an autoimmune condition.
